SEQUENCE LISTING (28 sequences)

SEQ ID NO: 1 - protein, 10 aa, Homo sapiens
GEAVAARKLF

SEQ ID NO: 2 - protein, 10 aa, Homo sapiens
NCPVPGKPGE

SEQ ID NO: 3 - protein, 10 aa, Homo sapiens
PVPGKPGEAV

SEQ ID NO: 4 - protein, 12 aa, Homo sapiens
NCPVPGKPGEAV

SEQ ID NO: 5 - protein, 5 aa, synthetic construct
  REGION 1..5: note = mAb VH CDR1
DYEMH

SEQ ID NO: 6 - protein, 17 aa, synthetic construct
  REGION 1..17: note = mAb VH CDR2
AIHPGYGGTAYNQKFKG

SEQ ID NO: 7 - protein, 7 aa, synthetic construct
  REGION 1..7: note = mAb VH CDR3
FITKFDY

SEQ ID NO: 8 - protein, 16 aa, synthetic construct
  REGION 1..16: note = mAb VL CDR1
KSSQSLLDSDGKTFLN

SEQ ID NO: 9 - protein, 7 aa, synthetic construct
  REGION 1..7: note = mAb VL CDR2
LVSKLDS

SEQ ID NO: 10 - protein, 9 aa, synthetic construct
  REGION 1..9: note = mAb VL CDR3
WQGTHFPWT

SEQ ID NO: 11 - protein, 17 aa, synthetic construct
  REGION 1..17: note = mAb VH CDR2
AILPGSGGTAYNQKFKG

SEQ ID NO: 12 - protein, 7 aa, synthetic construct
  REGION 1..7: note = mAb VH CDR3
LITTFDY

SEQ ID NO: 13 - protein, 16 aa, synthetic construct
  REGION 1..16: note = mAb VL CDR1
KSSQSLLDSDGKTYLN

SEQ ID NO: 14 - protein, 6 aa, synthetic construct
  REGION 1..6: note = mAb VH CDR1
SGYSWH

SEQ ID NO: 15 - protein, 16 aa, synthetic construct
  REGION 1..16: note = mAb VH CDR2
YIHYSGSTTYNPSLKG

SEQ ID NO: 16 - protein, 10 aa, synthetic construct
  REGION 1..10: note = mAb VH CDR3
WGTGHWYFDV

SEQ ID NO: 17 - protein, 14 aa, synthetic construct
  REGION 1..14: note = mAb VL CDR1
RSSTGAVTTTNYAN

SEQ ID NO: 18 - protein, 7 aa, synthetic construct
  REGION 1..7: note = mAb VL CDR2
GTNNRVP

SEQ ID NO: 19 - protein, 9 aa, synthetic construct
  REGION 1..9: note = mAb VL CDR3
ALWYSNHWV

SEQ ID NO: 20 - protein, 15 aa, Homo sapiens
KPGEAVAARKLFAPQ

SEQ ID NO: 21 - protein, 23 aa, Homo sapiens
AYTKRLGTEKEVEVIGGADKYHS

SEQ ID NO: 22 - protein, 185 aa, synthetic construct
  REGION 1..185: note = mAb VH
METGLRWLLLVAVLKGVQCQEQLEESGGDLVKPEGSLTLTCTASRFSFSSSYWICWVRQAPGKGLEWIACIYAGDSGSSYYASWAKGRFTVSKTSSTTVTLQTTSLTAADTATYFCARASVGAAYDYFALWGPGTLVTVSSGQPKAPSVFPLAPCCGDTPSSTVTLGCLVKGYLPEPVTVTWNSG

SEQ ID NO: 23 - protein, 236 aa, synthetic construct
  REGION 1..236: note = mAb VL
MDTRAPTQLLGLLLLWLPGARCALVMTQTPASVEAAMGGTVTIKCQASEDVSSHLAWYQQRPGQPPKLLIYGASDLASGVPSRFTGSGSGTQFTLAISDLECADAATYYCQGYYYISDSPYVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 24 - protein, 187 aa, synthetic construct
  REGION 1..187: note = mAb VH
METGLRWLLLVAVLKGVQCQSLEESGGGLVQPEGSLTLTCTASGFSFSSGYDMCWVRQTPGKGLEWIACISVDSDGVTYYASWAKGRFTISKTSSTTVTLQMTSLTAADTATYFCARGYESSSGVYIPYFTLWGPGTLVTVSSGQPKAPSVFPLAPCCGDTPSSTVTLGCLVKGYLPEPVTVTWNSG

SEQ ID NO: 25 - protein, 238 aa, synthetic construct
  REGION 1..238: note = mAb VL
MDMRAPTQLLGLLLLWLPGARCADIVLTQTPASVEAAVGGTVTIKCQASQSIYSYLAWYQHKPGQPPKLLIYKASTLASGVPSRFKGSGSGTEYTLTISDLECADAATYYCQHYYYSSTSGGGVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 26 - protein, 179 aa, synthetic construct
  REGION 1..179: note = mAb VH
METGLRWLLLVAVLKGVQCQSLEESGGRLVTPGTPLTLTCTASGFSLSNYYMSWVRQAPGKGLEWIGIIYGDDNTYCANWTKGRFTISKTSTTVDLTITSPTTEDTATYFCARGPDYIAAKMDIWGPGTLVTVSLGQPKAPSVFPLAPCCGDTPSSTVTLGCLVKGYLPEPVTVTWNSG

SEQ ID NO: 27 - protein, 239 aa, synthetic construct
  REGION 1..239: note = mAb VL
MDTRAPTQLLGLLLLWLPGARCDVVMTQTPASVEAAVGGTVTIKCQASQSISGYLSWYQQKPGQRPKLLIYRASTLESGVPSRFKGSGSGTEFTLTISDLECADAATYYCQCTYGSSTFSSYGNAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 28 - protein, 234 aa, Homo sapiens
MSCINLPTVLPGSPSKTRGQIQVILGPMFSGKSTELMRRVRRFQIAQYKCLVIKYAKDTRYSSSFCTHDRNTMEALPACLLRDVAQEALGVAVIGIDEGQFFPDIVEFCEAMANAGKTVIVAALDGTFQRKPFGAILNLVPLAESVVKLTAVCMECFREAAYTKRLGTEKEVEVIGGADKYHSVCRLCYFKKASGQPAGPDNKENCPVPGKPGEAVAARKLFAPQQILQCSPAN

(19) United States
(12) Patent Application Publication
(10) Pub. No.: US 2026/0235608 A1
(43) Pub. Date: Aug. 13, 2026

(54) OVARIAN CANCER RISK PREDICTION

(71) Applicant: AROCELL AB, Bromma (SE)

(72) Inventors: Kiran KUMAR JAGARLAMUDI, Uppsala (SE); Staffan ERIKSSON, Lidingö (SE); Josko OSREDKAR, Ljubljana (SI)

(21) Appl. No.: 19/157,039

(22) PCT Filed: Feb. 29, 2024
(86) PCT No.: PCT/SE2024/050195

§ 371 Date: Aug. 15, 2025

(30) Foreign Application Priority Data
Mar. 1, 2023 (SE) 2350229-7

(51) Int. Cl. G01N 33/57545

(52) U.S. Cl. CPC G01N 33/57545; G01N 2333/9122

(57) ABSTRACT

Ovarian cancer risk is predicted for a female human subject by determining an amount of STK1 material in a serum or plasma sample from the female human subject using first and second monoclonal antibodies, or antigen-binding fragments thereof, specifically binding to a serum form of human TK1. An amount of CA 125 or HE4 is determined in the serum or plasma sample or another serum or plasma sample from the female human subject. Ovarian cancer risk is then predicted for the female human subject based on the amount of STK1 material and the amount of one, but not both, of CA 125 and HE4.

### TECHNICAL FIELD

The present invention generally relates to measurement of thymidine kinase 1 (TK1), and in particular to predicting ovarian cancer risk based on measured amount of serum TK1 material.

### BACKGROUND

Ovarian cancer is the eighth most common cancer in women worldwide. The Global cancer observatory reports that over 30,000 new ovarian cancer cases and more than 20,000 ovarian cancer-related deaths occurred in the world in 2020. More than two-thirds of ovarian cancer patients are diagnosed in advanced stages of the disease (stage III or IV), which is associated with a 5-year survival of 27% for stage III and 13% for stage IV cancer patients. In contrast, the 5-year survival rate would improve significantly if ovarian cancer was detected in stage I, the 5-year survival in these cases is 90% and a 10-year survival 84%.

The most commonly used serum biomarker for ovarian cancer is carcinoma antigen 125 (CA 125), also known as mucin 16 (MUC16). Earlier studies showed that detection of ovarian cancer with the combination of CA 125 with pelvic ultrasonography or alone can be used for large cohort screening of populations. However, CA 125 have significant limitations in terms of sensitivity and specificity for detection of ovarian cancer in early stage [1]. Moreover, CA 125 levels is also elevated in different other pathological conditions, such as endometriosis and non-malignant gynecologic diseases [2].

In order to improve ovarian cancer detection other biomarkers, such as human epididymis protein 4 (HE4), also known as WAP four-disulfide core domain protein 2 (WFDC2), have been developed. HE4 is a glycoprotein belonging to the family of whey acidic four-disulfide core proteins, and it is overexpressed in serous and endometroid ovarian carcinomas. Studies have been conducted evaluating individual serum biomarkers for detection of ovarian carcinomas in women with pelvic masses as well as combination thereof. The combination of CA 125 with HE4 showed more efficient prediction of malignancy compared to either of them alone [3].

Furthermore, a dual marker algorithm based on CA 125 and HE4 was developed as a risk of ovarian malignancy algorithm (ROMA) that enhanced the clinical applications of these biomarkers in the differentiation of benign from malignant ovarian carcinomas [3-6]. However, another study demonstrated that there was no clinical benefit of using the ROMA index instead of CA 125 or HE4 alone in detection of epithelial ovarian carcinomas [7].

A new index in ovarian cancer diagnosis, risk of ovarian malignancy index (ROMI), has been developed based on ROMA but combining thymidine kinase 1 (TK1), HE4 and CA 125 [8]. ROMI was said to have better sensitivity and specificity as compared to ROMA in diagnosis of all-stage or stage I+II ovarian carcinoma. Also concluded that the combined detection of CA 125, HE4 and TK1 could significantly improve the sensitivity in the diagnosis of ovarian cancer.

There is still a need to improve the sensitivity and specificity for early detection as well as for differentiating benign pelvic masses from malignant ovarian tumors.

### SUMMARY

It is a general objective to provide an ovarian cancer risk prediction.

It is a particular objective to provide an ovarian cancer risk prediction capable of differentiating benign pelvic masses from malignant ovarian tumors.

It is another particular objective to provide an ovarian cancer risk prediction capable of differentiating early-stage of ovarian cancer (stage I, II) from advanced-stage of ovarian cancer (stage III, IV).

These and other objectives are met by embodiments as described herein.

The present invention is defined in the independent claim. Further embodiments of the invention are defined in the dependent claims.

An aspect of the invention relates to a method for predicting ovarian cancer risk for a female human subject. The method comprises determining an amount of serum thymidine kinase 1 (STK1) material in a serum or plasma sample from the female human subject using a kit comprising a first monoclonal antibody, or a first antigen-binding fragment thereof, specifically binding to a serum form of human TK1 and a second monoclonal antibody, or a second antigen-binding fragment thereof, specifically binding to the serum form of human TK1. The method also comprises determining an amount of carcinoma antigen 125 (CA 125) or human epididymis protein 4 (HE4) in the serum or plasma sample or another serum or plasma sample from the female human subject. The method further comprising predicting ovarian cancer risk for the female human subject based on the amount of STK1 material and the amount of one, but not both, of CA 125 and HE4.

Combination of STK1 with CA 125 or HE4 showed better performance than either of them alone in differentiation of early-stage ovarian cancer from the healthy control group, but also significantly better than STK1 in combination with ROMA index, i.e., STK1 with CA 125 and HE4. However, this was not observed using a TK1 activity test in combinations with the other markers. Furthermore, the combination of TK1 protein and CA 125 or HE4 could differentiate early-stage disease (stage I, II) more efficiently from advanced-stage (stage III, IV) disease.

### BRIEF DESCRIPTION OF THE DRAWINGS

The invention, together with further objects and advantages thereof, may best be understood by making reference to the following description taken together with the accompanying drawings, in which:

FIG. 1 TK1 levels in different groups, (A, B, C) serum TK1 protein (STK1p), (D, E, F) serum TK1 activity (STK1a), (A, D) all women, (B, E) premenopausal women, (C, F) postmenopausal women;

FIG. 2 ROC curves for (A) biomarkers STK1p, CA 125, HE4 alone, and as the dual markers with STK1p, and for (B) biomarkers STK1a, CA 125, HE4 alone, and as the dual markers with STK1a;

FIG. 3 The diagnostic performances of STK1p alone, STK1p+CA 125, STK1p+HE4, and STK1p+ROMA index for (A) premenopausal women, (B) postmenopausal women, (C) benign mass vs. healthy controls, (D) malignant ovarian cancer vs healthy controls, (E) benign mass vs malignant ovarian cancer;

FIG. 4 The combination of (A) STK1p+CA 125, (B) STK1a+CA 125, (D) STK1p+HE4, and (E) STK1a+HE4 and analysis of the (C, F) ROC curves in differentiation of early stages (I+II) from late stages (III+IV) malignant ovarian cancer;

FIG. 5 (A, B, C) STK1p and (D, E, F) STK1a levels before and after surgery for (A, D) all women, (B, E) pre-menopausal women, and (C, F) post-menopausal women; and

FIG. 6 (A) The ratio of STK1p at diagnosis/STK1p after surgery and with the number of days, (B) STK1p levels at diagnosis and after chemotherapy, (C) STK1p+CA 125, (D) STK1p+HE4, (E) STK1a+CA 125 and (F) STK1a+HE4 after chemotherapy with or without relapse.

### DETAILED DESCRIPTION

The present invention generally relates to measurement of thymidine kinase 1 (TK1), and in particular to predicting ovarian cancer risk based on measured amount of serum TK1 (STK1) material.

The success of ovarian cancer treatment is highly dependent on the time of detection of the disease, as patients with an early stage have the greatest chance of survival. Studies have shown that only <25% of patients with ovarian cancer can be diagnosed at the early stage when symptoms are not present, and 70% of patients are diagnosed at advanced stage despite considerable efforts aimed at early detection, no cost-effective screening test has so far been developed. Early detection with the help of tumor-specific biomarkers could, thus, improve the clinical outcome of patients with ovarian cancer. This is especially important for patients who have vague or no symptoms. In 2008, Moore et al. set up a mathematical algorithm for determining the risk of ovarian cancer (ROMA), which depends on the menopausal status of the woman and the preoperative levels of human epididymis protein 4 (HE4) and carcinoma antigen 125 (CA 125) in serum [6]. The ROMA index was, thus, designed with the aim of improving the usefulness of the tumor marker CA 125 in the diagnosis and monitoring of epithelial ovarian cancer. The sensitivity and specificity of the ROMA index were 63% and 95% as shown in the Example section. Still, this is not sufficient for efficient early detection of ovarian cancer.

Thus, there is a need for additional biomarker and results presented herein show that STK1 could be such a new diagnostic tool. The addition of STK1 to CA 125 or HE4 demonstrated a sensitivity above 70% with a specificity of 95%. Hence, the sensitivity for detection of early stage of ovarian cancer can be improved by combining STK1 with CA 125 or HE4.

The results show that higher sensitivity of combinations STK1 and CA 125, and STK1 and HE4 along with 95% specificity could offer early detection of ovarian cancer and therefore improve the prognosis of patients. In the differentiation of malignant ovarian cancer from healthy controls, the dual biomarkers STK1 and CA 125, and STK1 and HE4 showed higher sensitivity compared to STK1 alone. In the differentiation of benign from malignant ovarian cancer, the combination of STK1 and HE4 had a sensitivity of 58% followed by STK1 and CA 125 at 44% compared to STK1 at 28% alone. These results indicate that the combination of STK1p and CA 125 offers better diagnostic performance in the detection of early-stage ovarian cancer. In addition, a combination of STK1 with CA 125 as well as a combination of STK1 with HE4 significantly differentiate ovarian cancer patients based on stage and identify the probability of patients with tumor relapse.

Furthermore, the combination STK1 and CA 125 showed higher positive predictive value (PPV) and negative predictive value (NPV) than the combination STK1 and ROMA index (96% vs 95% and 80% vs 73%). In the differentiation of benign mass from healthy controls, the combination of STK1 and CA 125 had the highest sensitivity (64%) compared to the other combinations.

Hence, combining STK1 with one, but not both, of CA 125 and HE4 leads to improvement in ovarian cancer risk prediction over any of these biomarkers (STK1, CA 125, or HE4) alone or indeed over a combination of all three biomarkers, i.e., STK1, CA 125 and HE4 (STK1p+ROMA). This was highly surprising since the combination of TK1, CA 125 and HE4 has previously been suggested as a new risk of ovarian malignancy index (ROMI) that is said to have better sensitivity and specificity as compared to ROMA [8]. Thus, it was totally unexpected that even better ovarian cancer risk prediction could be achieved by omitting one of CA 125 and HE4 in the prediction so that the combination of STK1 and CA 125 or the combination of STK1 and HE4 achieves improved prediction capability as compared to the combination of STK1, CA 125 and HE4.

An aspect of the invention relates to a method for predicting ovarian caser risk for a female human subject. The method comprises determining an amount of STK1 material in a serum or plasma sample from the female human subject using a kit comprising a first monoclonal antibody, or a first antigen-binding fragment thereof, specifically binding to a serum form of human TK1 and a second monoclonal antibody, 20 or a second antigen-binding fragment thereof, specifically binding to the serum form of human TK1. The method also comprises determining an amount of CA 125 or HE4 in the serum or plasma sample or another serum or plasma sample from the female human subject. The method further comprises predicting ovarian cancer risk for the female subject based on the amount of STK1 material and the amount of one, but not both, of CA 125 and HE4.

TK1 proteins in humans are present in various forms depending on the presence of certain molecules, e.g., presence or absence of adenosine triphosphate (ATP); depending on the concentration of the protein, i.e., high or low concentration; depending on the type of the protein, i.e., native or recombinant TK1; and depending on the site of the protein, i.e., in serum or cytosol.

Generally, cytosolic and recombinant human TK1 occurs as tetramers in the presence of ATP or at high concentration, and as dimers in the absence of ATP or at low concentration. The tetramer form of cytosolic and recombinant human TK1 has high TK1 activity, whereas the dimer form has lower TK1 activity. Cytosolic TK1, also referred to as cellular TK1, is TK1 present inside cells and can be isolated from such cells.

Human STK1, in clear contrast, can be in the form of high molecular weight complexes, such as oligomers or comprising such oligomers, having TK1 activity and dimer and tetramer forms having very low or even lacking TK1 activity. The oligomerization seems to be related to the formation of disulfide cross linking occurring in the blood. STK1 is found in the blood of a patient and can thereby be determined in a plasma sample or a serum sample.

STK1 material as used herein refers to STK1 in its various forms, such as dimers, tetramers, oligomers and complexes comprising STK1. The STK1 material is present in blood, blood plasma or serum in subjects. The STK1 material may then comprise STK1 in the above-mentioned forms, such as dimers, tetramers, oligomers and complexes comprising STK1. STK1 material also includes complexes with at least one TK1 protein unit and other molecules and/or macromolecules.

In the art, various gene expressions arrays have been proposed to determine the TK1 messenger ribonucleic acid (mRNA) transcripts in cancer cell samples and biopsies. As mentioned above, TK1 is available in various forms in subjects, including cytosolic TK1 and serum TK1. Gene expression arrays determining TK1 mRNA transcripts from such biopsy samples are mainly assaying TK1 mRNA transcripts of cytosolic TK1 present in cancer cells. Hence, such gene expression arrays cannot be used to determine the level of STK1 material in a female human subject.

In an embodiment, determining the amount of STK1 material comprises contacting the serum or plasma sample with the first monoclonal antibody, or the first antigen-binding fragment thereof, and the second monoclonal antibody, or the second antigen-binding fragment thereof. This embodiment also comprises measuring an amount of the first monoclonal antibody, or the first antigen-binding fragment thereof, or the second monoclonal antibody, or the second antigen-binding fragment thereof, bound to the STK1 material.

Contacting the serum or plasma sample with the first and second monoclonal antibodies, or the first and second antigen-binding fragments thereof, may be achieved by adding the first and second antibodies, or the first and second antigen-binding fragments thereof, to the serum or plasma sample and incubating the serum or plasma sample with the first and second monoclonal antibodies, or the first and second antigen-binding fragments thereof. The first and second monoclonal antibodies, or the first and second antigen-binding fragments thereof, thereby bind to the STK1 material forming a complex between the first and second monoclonal antibodies, or the first and second antigen-binding fragments thereof, and the STK1 material. In such an embodiment, measuring the amount of the first or second monoclonal antibody, or the first or second antigen-binding fragment thereof, bound to the STK1 material can include measuring or quantifying the complex between the first and second monoclonal antibodies, or the first and second antigen-binding fragments thereof, and the STK1 material to thereby measure or quantify the amount of the first or second monoclonal antibody, or the first or second antigen-binding fragment thereof, bound to the STK1 material.

In an embodiment, the method also comprises correlating the measured amount of the first or second monoclonal antibody, or the first or second antigen-binding fragment thereof, bound to the STK1 material to an amount of STK1 material. This may be performed using a pre-defined correlation between measured amount of the first or second monoclonal antibody, or the first or second antigen-binding fragment thereof, bound to a reference TK1 material and concentration of the reference TK1 material. A typical reference TK1 material that can be used when generating such a pre-defined correlation is recombinant human TK1.

The pre-defined correlation may, thus, be generated by adding or contacting the first and second monoclonal antibodies, or the first and second antigen-binding fragments thereof, to different samples comprising different concentrations of the reference TK1 material, preferably recombinant human TK1. The amount of the first or second monoclonal antibody, or the first or second antigen-binding fragment thereof, bound to the reference TK1 material, preferably recombinant human TK1, is then measured in the different samples to thereby get a standard curve, function or relationship between concentration of reference TK1 material, preferably recombinant human TK1, and the measured amount of the first or second monoclonal antibody, or the first or second antigen-binding fragment thereof, bound to the reference TK1 material, preferably recombinant human TK1.

This pre-defined correlation, such as standard curve, function or relationship, can then be used to map or convert the measured amount of the first or second monoclonal antibody, or the first and second antigen-binding fragment thereof, bound to the STK1 material in the serum or plasma sample to a concentration of the STK1 material in the serum or plasma sample.

It is generally preferred if the same kit and the same types of first and second monoclonal antibodies, or first and second antigen-binding fragments thereof, are used for generating the pre-defined correlation as for determining an amount of STK1 material in a serum or plasma sample from the female human subject. Hence, in a preferred embodiment, the first and second monoclonal antibodies, or the first and second antigen-binding fragments thereof, are capable of specifically binding to not only the serum form of human TK1 but also to the reference TK1 material, preferably recombinant human TK1.

In an embodiment, the serum or plasma sample is processed prior to or during the incubation of the serum or plasma sample with the first and second monoclonal antibodies, or the first and second antigen-binding fragments thereof. This sample processing may be used to stabilize selected STK1 forms in the serum or plasma sample and/or to break larger STK1 complexes or oligomers into smaller complexes or multimers.

Hence, in an embodiment, a sample dilution or pretreatment buffer is added to the serum or plasma sample, preferably prior to or in connection with adding the first and second antibodies, or the first and second antigen-binding fragments thereof, to the serum or plasma sample, preferably prior to adding the antibody, or the antigen-binding fragment thereof, to the serum or plasma sample.

In an embodiment, the sample dilution buffer comprises ATP, preferably in a concentration selected within an interval of from 0.5 mM up to 50 mM, such as from 0.5 mM up to 20 mM or from 1.5 mM up to 50 mM. As previously described herein, ATP stabilizes the tetramer form of TK1, which has high enzymatic TK1 activity.

In another embodiment, the sample dilution buffer comprises a reducing agent. The reducing agent may then break disulfide cross links in larger STK1 complexes and oligomers to obtain smaller STK1 forms, such as tetramers. Various reducing agents capable of breaking disulfide bonds can be used according to the embodiments including, but not limited to, dithioerythritol (DTE), dithiothreitol (DTT), dithiobutylamin (DTBA), tris(2-carboxyethyl) phosphine) (TCEP), and combinations thereof. The amount of the reducing agent is typically selected within an interval of from 0.1 mM up to 10 mM.

The sample dilution buffer may, in an embodiment, comprise both ATP and a reducing agent.

Hence, in an embodiment, the method comprises adding a sample dilution buffer to the serum or plasma sample. The sample dilution buffer comprises, in this embodiment, ATP and a reducing agent. In a particular embodiment, the sample dilution buffer comprises ATP in a concentration selected within an interval of from 0.5 mM up to 50 mM, preferably selected within an interval of from 0.5 mM up to 20 mM, and more preferably selected within an interval of from 1.5 mM up to 50 mM. In a particular embodiment, the reducing agent is selected from the group consisting of dithioerythritol (DTE), dithiothreitol (DTT), dithiobutylamin (DTBA), tris(2-carboxyethyl) phosphine) (TCEP), and any combination thereof. In a particular embodiment, the sample dilution buffer comprises the reducing agent in a concentration selected within an interval of from 0.1 mM up to 10 mM.

In an embodiment, the amount of STK1 material is determined using the first and second monoclonal antibodies, or the first and second antigen-binding fragments thereof, specifically binding to the serum form of human TK1 in the serum or plasma sample taken from the female human following treatment for ovarian cancer. The amount of CA 125 or HE4 is determined, in this embodiment, in the serum or plasma sample or another serum or plasma sample taken from the female subject following treatment for ovarian cancer. In this embodiment, the method comprises predicting relapse of ovarian cancer for the female subject based on the amount of STK1 material and the amount of one, but not both, of CA 125 and HE4.

Hence, in this embodiment, the ovarian cancer risk prediction comprises predicting relapse of ovarian cancer, i.e., predicting a risk of relapse of ovarian cancer for a female subject treated for ovarian cancer. Treatment of ovarian cancer typically involves surgery, and in particular removal of the uterus, i.e., hysterectomy, and sometimes also along with one or both ovaries and fallopian tubes, i.e., bilateral salpingo-oophorectomy (BSO). Alternatively, or in addition, the treatment of ovarian cancer can involve chemotherapy.

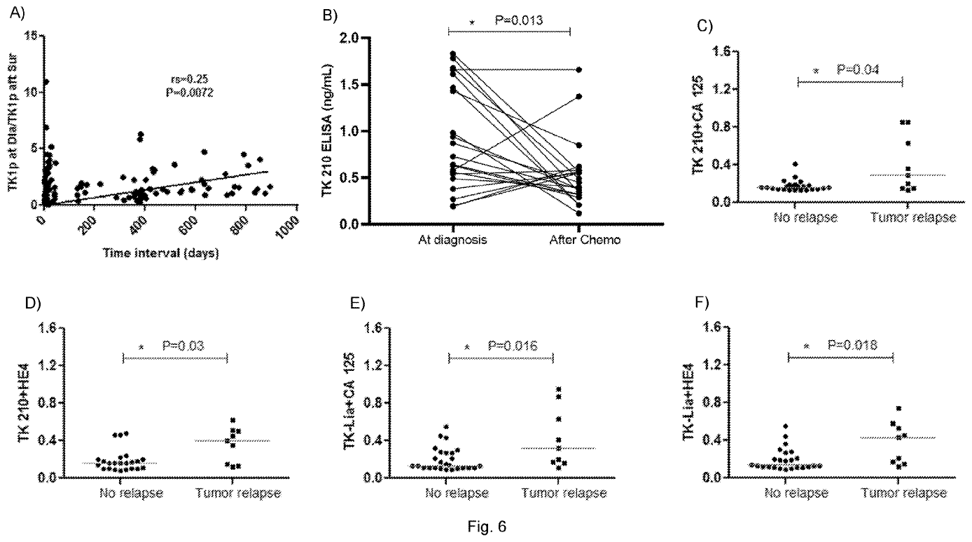

As shown herein, the patients with relapse after treatment, such as chemotherapy, had significantly higher levels of STK1+CA 125 as well as STK1+HE4 compared to patients without relapse (FIGS. 6C and 6D).

In an embodiment, the amount of STK1 material is determined using the first and second monoclonal antibodies, or the first and second antigen-binding fragments thereof, specifically binding to the serum form of human TK1 in a serum or plasma sample taken from the female subject prior to treatment for ovarian cancer, such as in connection with diagnosis of ovarian cancer. In this embodiment, the amount of CA 125 or HE4 is determined in the serum or plasma sample or another serum or plasma sample taken form the female subject prior to treatment for ovarian cancer, such as in connection with diagnosis of ovarian cancer. Prediction of relapse of ovarian cancer is then preferably predicted for the female subject based on the amount of STK1 material and the amount of one, but not both, of CA 125 and HE4 determined in the serum or plasma sample(s) taken form the female subject prior to treatment for ovarian cancer and the amount of STK1 material and the amount of one, but not both, of CA 125 and HE4 determined in the serum or plasma sample(s) taken form the female subject following treatment for ovarian cancer.

In an embodiment, predicting ovarian cancer risk comprises predicting an ovarian cancer stage for the female human subject based on the amount of STK1 material and the amount of one, but not both, of CA 125 and HE4.

For, instance, the method may comprise predicting whether the female human subject is suffering from a FIGO stage I or II ovarian cancer or a FIGO stage III or IV stage ovarian cancer based on the amount of STK1 material and the amount of one, but not both, of CA 125 and HE4.

Generally, FIGO stage I means that the cancer is confined to one or both ovaries. Sometimes the FIGO I stage is divided into stage IA with growth limited to one ovary with no tumor on external surfaces, stage IB with growth limited to both ovaries with no tumor on external surfaces, and stage IC with tumor is either stage IA or IB but with tumor on surface of one or both ovaries. FIGO stage II means that the cancer has spread to the uterus or other nearby organs. Sometimes the FIGO II stage is divided into stage IIA with extension and/or metastases of cancer to the uterus and/or fallopian tubes, stage IIB with extension of the cancer to other pelvic tissues, and stage IIC with tumor is at either stage IIA or IIB but with tumor on surface of one or both ovaries. FIGO stage III means that the cancer has spread to the lymph nodes or abdominal lining. Sometimes the FIGO III stage is divided into stage IIIA with tumor limited to the true pelvis, stage IIIB with metastasis of abdominal peritoneal surfaces≤2 cm in diameter and stage IIIC with peritoneal metastasis beyond the pelvis >2 cm in diameter. FIGO stage IV means that the cancer has spread to distant organs, such as the lungs or liver.

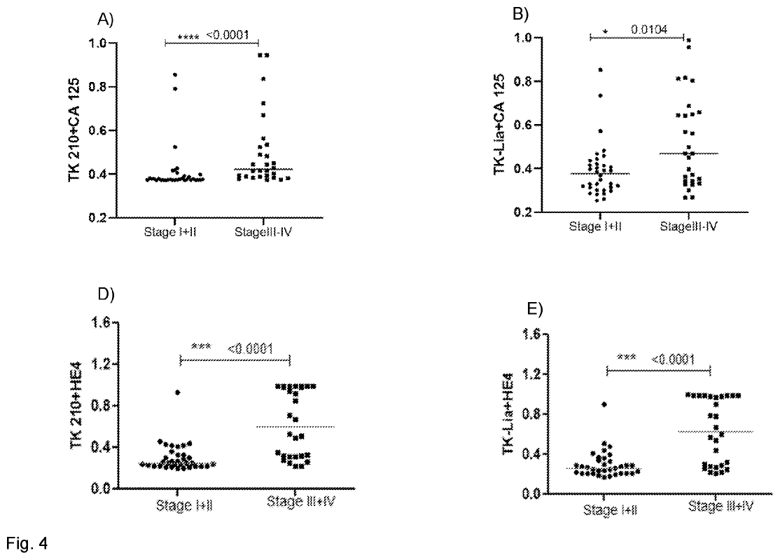

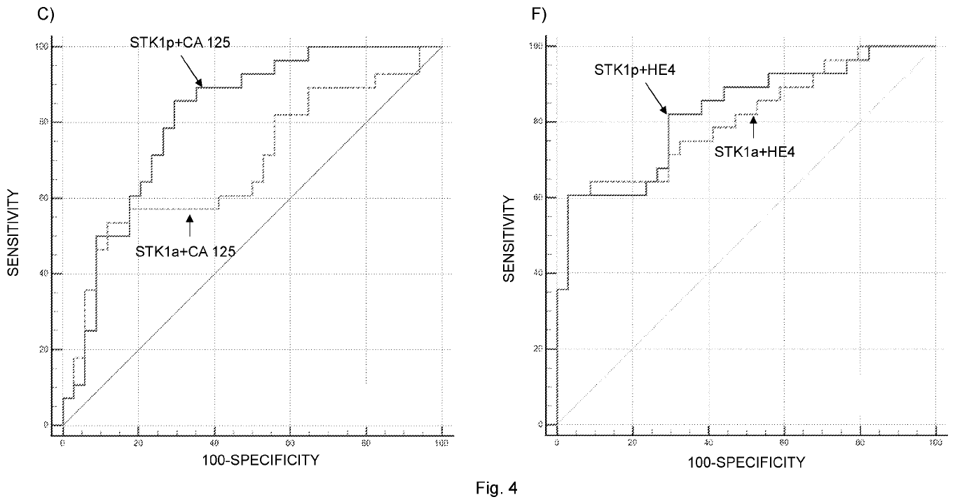

As shown herein, the combination of STK1 and CA 125 was significantly higher in patients in FIGO stage III+IV as compared to patients in FIGO stage I+II (FIG. 4A). A similar result was obtained with the combination of STK1 and HE4 (FIG. 4D).

In a particular embodiment, the female human subject is in a premenopausal status. Hence, in an embodiment, the female subject is in a premenopausal status when taking the serum or plasma sample(s) from the female human subject.

Experimental data as used herein indicate that the combination of the amount of STK1 material and the amount of one, but not both, of CA 125 and HE4 was in particular useful in detection of all stages of cancer, in the detection of benign tumor mass from healthy controls, in the differentiation of malignant ovarian cancer from healthy controls and in the differentiation of benign mass from malignant ovarian cancer for female human subjects in premenopausal status as compared to in postmenopausal status.

In a particular embodiment, the method comprises determining an amount of CA 125 in the serum or plasma sample or the another serum or plasma sample from the female human subject. In this particular embodiment, the method comprises predicting ovarian cancer risk for the female human subject based on the amount of STK1 material and the amount of CA 125 but not on amount of HE4.

Hence, in a particular embodiment, the ovarian cancer risk prediction for the female human subject is based solely on the amount of STK1 material and the amount of CA 125.

As shown herein the combination of STK1 and CA 125 gives the best diagnostic performance in the detection of early-stage ovarian cancer in comparison with the healthy group. For premenopausal women, the combination of STK1 and CA 125 showed higher PPV and NPV than the combination of STK1 and ROMA index (96% vs 95% and 80% vs 73%). The results strongly indicate that the dual biomarker STK1 and CA 125 had the best diagnostic performance for the detection of benign as well as malignant ovarian cancers in comparison to the healthy control group.

In another particular embodiment, the method comprises determining an amount of HE4 in the serum or plasma sample or the another serum or plasma sample from the female human subject. In this particular embodiment, the method comprises predicting ovarian cancer risk for the female human subject based on the amount of STK1 material and the amount of HE4 but not on amount of CA 125.

Hence, in a particular embodiment, the ovarian cancer risk prediction for the female human subject is based solely on the amount of STK1 material and the amount of HE4.

In an embodiment, a dual biomarker index can be calculated based on the determined amount of STK1 material and the amount of CA 125 or based on the determined amount of STK1 and the amount of HE4.

The dual biomarker indices can be calculated according to various embodiments, i.e., dual biomarker index 1 (In 1)=f (STK1, CA 125) and dual biomarker index 2 (In 2)=g (STK1, HE4), wherein STK1 represents the amount of STK1 material, CA 125 represents the amount of CA 125, HE4 represents the amount of HE4 and f( ) and g( ) are functions. As an example, the dual biomarker indices In 1 and In 2 can be calculated using logistic regression of STK1 and CA 125 or of STK1 and HE4. For instance, such logistic regression can be done using MedCalc version 17.6.

Generally, logistic regression generates coefficients (and its standard errors and significance levels) of a formula to predict a logit transformation of the probability of the presence of a characteristic of interest (COI):

$$Logit ( p ) = {b}_{0} + {b}_{1} {X}_{1 +} {b}_{2} {X}_{2 +} {b}_{3} {X}_{3} + … {b}_{k} {X}_{k}$$

where p is the probability of the presence of the COI. The logit transformation is defined as the logged odds, odds=p/(1−p), which represents the probability of the presence of the COI divided by the probability of absence of COI, so that logit (p)=In (p/1−p).

Such logistic regression was used to calculate In 1 and In 2 for the serum samples in the Example. The In 1 and In 2 values were in the range of 0 to 1.0. The median In 1 value of the healthy group was 0.23 and the median In 1 value of ovarian cancer patients (including benign and malignant) was 0.98. The corresponding median values of In 2 were 0.26 for the healthy group and 0.95 for the ovarian cancer patients.

The In 1 and In 2 values can be used as described herein, such as for early-stage diagnosis of ovarian cancer. For instance, In 1 and In 2 values between 0 to 0.70 indicate healthy (95% CI), i.e., low risk for ovarian cancer, whereas values of In 1 and In 2 between 0.70 to 1.00 indicate an ovarian cancer patient, i.e., high risk for ovarian cancer.

For differentiation of benign from malignant, In 1 and In 2 values below 0.60 indicate benign (95% CI) and In 1 and In 2 values between 0.60 to 1.00 indicate malignant ovarian cancer.

Correspondingly, to differentiate early stages (stage I+II) of ovarian cancer from the late stages (stage III+IV) of ovarian cancer, values from 0 to 0.40 of In 1 and In 2 indicate early stages of ovarian cancer and value above 0.40 to 1.00 indicate late stages of ovarian cancer.

The first and second monoclonal antibodies, or the first and second antigen-binding fragments thereof, specifically bind to the STK1 material, and in particular bind specifically to the serum form of the TK1 protein.

The specificity of an antibody, or an antigen-binding fragment thereof, can be determined based on affinity and/or avidity. The affinity, represented by the equilibrium constant for the dissociation (K_{d}) of an antigen with the antibody, or the antigen-binding fragment thereof, is a measure for the binding strength between an antigenic determinant and an antigen-binding site on the antibody, or the antigen-binding fragment thereof. The lesser the value of K_{d}, the stronger the binding strength between the antigenic determinant and the antibody, or the antigen-binding fragment thereof. Alternatively, the affinity can also be expressed as the affinity constant (Kₐ), which is 1/K_{d}. As will be clear to the skilled person, affinity can be determined in a manner known per se, depending on the specific antigen of interest.

Avidity is the measure of the strength of binding between an antibody, or an antigen-binding fragment thereof, and the pertinent antigen. Avidity is related to both the affinity between an antigenic determinant and its antigen binding site on the antibody, or the antigen-binding fragment thereof, and the number of pertinent binding sites present on the antibody, or the antigen-binding fragment thereof.

Typically, antibodies, or antigen-binding fragments thereof, will bind to their antigen with a dissociation constant (K_{d}) of 10⁻⁵ to 10⁻¹² moles/liter (M) or less, and preferably 10⁻⁷ to 10⁻¹² M or less and more preferably 10⁻⁸ to 10⁻¹² M, i.e., with an association constant (Kₐ) of 10⁵ to 10¹² M⁻¹ or more, and preferably 10⁷ to 10¹² M⁻¹ or more and more preferably 10⁸ to 10¹² M⁻¹.

Generally, any K_{d} value greater than 10⁻⁴ M (or any Kₐ value lower than 10⁴ M⁻¹) is generally considered to indicate non-specific binding. Preferably, an antibody, or an antigen-binding fragment thereof, will bind to the STK1 material with an affinity less than 500 nM, preferably less than 200 nM, more preferably less than 10 nM, such as less than 5 nM or even lower, such as 1 nM or lower.

Specific binding of an antibody, or an antigen-binding fragment thereof, to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.

In an embodiment, at least one of the first and second antibodies, or the antigen-binding fragments thereof, has specificity for an epitope or peptide consisting of an amino acid sequence from the C-terminal region of human TK1.

The peptide is preferably selected from a portion of TK1 ranging from amino acid position 200 to the end of the TK1, i.e., amino acid position 234 in humans (SEQ ID NO: 28). In a particular embodiment, the peptide is selected from a portion of the TK1 protein ranging from amino acid position 205, preferably 210 to amino acid position 230, preferably 225.

The peptide is preferably an N-mer, wherein N is an integer within a range of from 8 up to 20, preferably within a range of from 10 up to 15. The peptide preferably consists of N consecutive amino acids in the C-terminal region of the TK1 protein.

In an embodiment, the peptide consists of the following amino acid sequence GEAVAARKLF (SEQ ID NO: 1). In another embodiment, the peptide consists of the following amino acid sequence NCPVPGKPGE (SEQ ID NO: 2). In a further embodiment, the peptide consists of the following amino acid sequence PVPGKPGEAV (SEQ ID NO: 3). In yet another embodiment, the peptide consists of the following amino acid sequence NCPVPGKPGEAV (SEQ ID NO: 4).

A monoclonal antibody having specificity for an epitope consisting of GEAVAARKLF (SEQ ID NO: 1) has a variable heavy (VH) domain complementarity determining region 1 (CDR1) having amino acid sequence DYEMH (SEQ ID NO: 5), a VH domain CDR2 having amino acid sequence AIHPGYGGTAYNQKFKG (SEQ ID NO: 6), a VH domain CDR3 having amino acid sequence FITKFDY (SEQ ID NO: 7), a variable light (VL) domain CDR1 having amino acid sequence KSSQSLLDSDGKTFLN (SEQ ID NO: 8), a VL domain CDR2 having amino acid sequence LVSKLDS (SEQ ID NO: 9) and a VL domain CDR3 having amino acid sequence WQGTHFPWT (SEQ ID NO: 10).

A monoclonal antibody having specificity for the epitopes NCPVPGKPGE (SEQ ID NO: 2), PVPGKPGEAV (SEQ ID NO: 3) and NCPVPGKPGEAV (SEQ ID NO: 4) has a VH domain CDR1 having amino acid sequence DYEMH (SEQ ID NO: 5), a VH domain CDR2 having amino acid sequence AILPGSGGTAYNQKFKG (SEQ ID NO: 11), a VH domain CDR3 having amino acid sequence LITTFDY (SEQ ID NO: 12), a VL domain CDR1 having amino acid sequence KSSQSLLDSDGKTYLN (SEQ ID NO: 13), a VL domain CDR2 having amino acid sequence LVSKLDS (SEQ ID NO: 9), and a VL domain CDR3 having amino acid sequence WQGTHFPWT (SEQ ID NO: 10).

In another embodiment, one of the first and second antibodies, or the first and second antigen-binding fragments thereof, has specificity for a conformation dependent epitope of human TK1. A monoclonal antibody having specificity for such a conformation dependent epitope has a VH domain CDR1 having amino acid sequence SGYSWH (SEQ ID NO: 14), a VH domain CDR2 having amino acid sequence YIHYSGSTTYNPSLKG (SEQ ID NO: 15), a VH domain CDR3 having amino acid sequence WGTGHWYFDV (SEQ ID NO: 16), a VL domain CDR1 having amino acid sequence RSSTGAVTTTNYAN (SEQ ID NO: 17), a VL domain CDR2 having amino acid sequence GTNNRVP (SEQ ID NO: 18), and a VL domain CDR3 having amino acid sequence ALWYSNHWV (SEQ ID NO: 19).

The above-three presented examples of monoclonal anti-TK1 antibodies that can be used according to the embodiments are further disclosed in WO 2015/094106, the teaching of which regarding monoclonal anti-TK1 antibodies is incorporated herein by reference.

Hence, in an embodiment, at least one of the first and second monoclonal antibodies, or the first and second antigen-binding fragments thereof, is selected from the group consisting of a monoclonal antibody, or an antigen-binding fragment thereof, having specificity for GEAVAARKLF (SEQ ID NO: 1) of human TK1, a monoclonal antibody, or an antigen-binding fragment thereof, having specificity for at least one of NCPVPGKPGE (SEQ ID NO: 2), PVPGKPGEAV (SEQ ID NO: 3) and NCPVPGKPGEAV (SEQ ID NO: 4) of human TK1, and a monoclonal antibody, or an antigen-binding fragment thereof, having specificity for a conformation dependent epitope of human TK1.

In another embodiment, one of the first and second monoclonal antibodies, or the first and second antigen-binding fragments thereof, has specificity for an epitope or peptide consisting of KPGEAVAARKLFAPQ (SEQ ID NO: 20). At least one additional amino acid, such as a cysteine residue, may be added to the N-terminal or C-terminal, preferably the N-terminal, of the peptide for use as coupling to other molecules, such as carrier proteins.

An antibody having specificity for this epitope is further disclosed in WO 95/29192, the teaching of which regarding anti-TK1 antibodies is incorporated herein by reference.

In a further embodiment, one of the first and second monoclonal antibodies, or the first and second antigen-binding fragments thereof, has specificity for an epitope or peptide consisting of an amino acid sequence from an active site of TK1. The peptide is preferably selected from a portion of TK1 ranging from amino acid position 150 to amino acid position 190 in human TK1. In a particular embodiment, the peptide is selected from a portion of TK1 ranging from amino acid position 155, preferably 160 and more preferably 161, to amino acid position 185, preferably 183.

The peptide is preferably an M-mer, wherein M is an integer within a range of from 10 up to 40, preferably within a range from 20 up to 30 and more preferably 23 or 24. The peptide preferably consists of M consecutive amino acids in the active site of the TK1 protein.

At least one additional amino acid, such as a cysteine residue, may be added to the N-terminal or C-terminal, preferably the N-terminal, of the peptide for use as coupling to other molecules, such as carrier proteins.

In an embodiment, the peptide consisting of an amino acid sequence from the active site of TK1 has an amino acid sequence corresponding to amino acid positions 161 to 183 in human TK1, i.e., has amino acid sequence of AYTKRLGTEKEVEVIGGADKYHS (SEQ ID NO: 21).

An antibody having specificity for this epitope is further disclosed in WO 2008/142664, the teaching of which regarding anti-TK1 antibodies is incorporated herein by reference.

In a further embodiment, at least one of the first and second monoclonal antibodies, or the first and second antigen-binding fragments thereof, is a monoclonal antibody or a fragment thereof as disclosed in WO 2019/201901, the teaching of which regarding monoclonal anti-TK1 antibodies is incorporated herein by reference.

For instance, the monoclonal antibody could be mAb 6C6, mAb 4H4 or mAb 23C11.

mAb 6C6 VH domain (SEQ ID NO: 22):
METGLRWLLLVAVLKGVQCQEQLEESGGDLVKPEGSLTLTCTASRFSFSSSYWICWVRQAPGKGLEWI
ACIYAGDSGSSYYASWAKGRFTVSKTSSTTVTLQTTSLTAADTATYFCARASVGAAYDYFALWGPGTL
VTVSSGQPKAPSVFPLAPCCGDTPSSTVTLGCLVKGYLPEPVTVTWNSG
mAb 6C6 VL domain (SEQ ID NO: 23):
MDTRAPTQLLGLLLLWLPGARCALVMTQTPASVEAAMGGTVTIKCQASEDVSSHLAWYQQRPGQPPK
LLIYGASDLASGVPSRFTGSGSGTQFTLAISDLECADAATYYCQGYYYISDSPYVFGGGTEVVVKGDPV
APTVLIFPPAADQVATGTVTIVCVANKYFPDVTVTWEVDGTTQTTGIENSKTPQNSADCTYNLSSTLTLT
STQYNSHKEYTCKVTQGTTSWQSFNRGDC
mAb 4H4 VH domain (SEQ ID NO: 24):
METGLRWLLLVAVLKGVQCQSLEESGGGLVQPEGSLTLTCTASGFSFSSGYDMCWVRQTPGKGLEWI
ACISVDSDGVTYYASWAKGRFTISKTSSTTVTLQMTSLTAADTATYFCARGYESSSGVYIPYFTLWGPG
TLVTVSSGQPKAPSVFPLAPCCGDTPSSTVTLGCLVKGYLPEPVTVTWNSG
mAb 4H4 VL domain (SEQ ID NO: 25):
MDMRAPTQLLGLLLLWLPGARCADIVLTQTPASVEAAVGGTVTIKCQASQSIYSYLAWYQHKPGQPPKL
LIYKASTLASGVPSRFKGSGSGTEYTLTISDLECADAATYYCQHYYYSSTSGGGVFGGGTEVVVKGDPV
APTVLIFPPAADQVATGTVTIVCVANKYFPDVTVTWEVDGTTQTTGIENSKTPQNSADCTYNLSSTLTLT
STQYNSHKEYTCKVTQGTTSVVQSFNRGDC
mAb 23C11 VH domain (SEQ ID NO: 26):
METGLRWLLLVAVLKGVQCQSLEESGGRLVTPGTPLTLTCTASGFSLSNYYMSWVRQAPGKGLEWIGII
YGDDNTYCANWTKGRFTISKTSTTVDLTITSPTTEDTATYFCARGPDYIAAKMDIWGPGTLVTVSLGQPK
APSVFPLAPCCGDTPSSTVTLGCLVKGYLPEPVTVTWNSG
mAv 23C11 VL domain (SEQ ID NO: 27):
MDTRAPTQLLGLLLLWLPGARCDVVMTQTPASVEAAVGGTVTIKCQASQSISGYLSWYQQKPGQRPKL
LIYRASTLESGVPSRFKGSGSGTEFTLTISDLECADAATYYCQCTYGSSTFSSYGNAFGGGTEVVVKGD
PVAPTVLIFPPAADQVATGTVTIVCVANKYFPDVTVTWEVDGTTQTTGIENSKTPQNSADCTYNLSSTLT
LTSTQYNSHKEYTCKVTQGTTSWQSFNRGDC

In an embodiment, one of the first monoclonal antibody, or the first antigen-binding fragment thereof, and the second monoclonal antibody, or the second antigen-binding fragment thereof, has specificity for a peptide consisting of an amino acid sequence from the C-terminal region of TK1. In this embodiment, the other of the first monoclonal antibody, or the first antigen-binding fragment thereof, and the second monoclonal antibody, or the second antigen-binding fragment thereof, has specificity for a peptide selected from the group consisting of a peptide consisting of the amino acid sequence from the C-terminal region of TK1, a peptide consisting of another amino acid sequence from the C-terminal region of TK1 and a peptide consisting of an amino acid sequence from the active site of TK1. In a particular embodiment, the other of the first monoclonal antibody, or the first antigen-binding fragment thereof, and the second monoclonal antibody, or the second antigen-binding fragment thereof, has specificity for a peptide consisting of another amino acid sequence from the C-terminal region of TK1.

According to the invention, the amount of STK1 material in the serum or plasma sample is determined using a kit. The kit comprises a first monoclonal antibody, or a first antigen-binding fragment thereof, and a second monoclonal antibody, or a second antigen-binding fragment thereof. The first and second monoclonal antibodies can be selected from the above-described illustrative examples of monoclonal anti-TK1 antibodies.

In a particular embodiment, the kit comprises a first monoclonal antibody, or a first antigen-binding fragment thereof, having specificity for an epitope selected from the group consisting of i) GEAVAARKLF (SEQ ID NO: 1) of human TK1, ii) at least one of NCPVPGKPGE (SEQ ID NO: 2), PVPGKPGEAV (SEQ ID NO: 3) and NCPVPGKPGEAV (SEQ ID NO: 4) of human TK1, and iii) a conformation dependent epitope of human TK1. The kit also comprises a second monoclonal antibody, or a second antigen-binding fragment thereof, having specificity for an epitope selected from the group consisting of i) GEAVAARKLF (SEQ ID NO: 1) of human TK1, ii) at least one of NCPVPGKPGE (SEQ ID NO: 2), PVPGKPGEAV (SEQ ID NO: 3) and NCPVPGKPGEAV (SEQ ID NO: 4) of human TK1, and iii) a conformation dependent epitope of human TK1.

In an embodiment, the first monoclonal antibody, or the first antigen-binding fragment thereof, is a so-called capture antibody immobilized to a support or intended to be immobilized to the support and the second monoclonal antibody, or the second antigen-binding fragment thereof, is a so-called detection antibody. In another embodiment, the second monoclonal antibody, or the second antigen-binding fragment thereof, is the capture antibody immobilized to the support or intended to be immobilized to the support, whereas the first monoclonal antibody, or the first antigen-binding fragment thereof, is used as detection antibody.

In an embodiment, the first and second monoclonal antibodies, or the first and second antigen-binding fragments thereof, have specificities for different epitopes in the STK1 material.

In another embodiment, the first and second monoclonal antibodies, or the first and second antigen-binding fragments thereof, have specificities for the same epitope in the STK1 material. This is possible since the same epitope may be present at multiple copies in the high molecular weight complexes of multiple TK1 protein units. Thus, the STK1 material may be a multivalent complex of multiple, i.e., at least two, TK1 protein units. In fact, the same type of monoclonal antibody, or antigen-binding fragment thereof, could be used as the first and second monoclonal antibody, or first and second antigen-binding fragment thereof.

In an embodiment, one of the first and the second monoclonal antibody, or the first and second antigen-binding fragment thereof, has specificity for a peptide consisting of an amino acid sequence from the active site of TK1 and the other of the first and second monoclonal antibody, or the first and second antigen-binding fragment thereof, has specificity for a peptide consisting of an amino acid sequence from the C-terminal region of TK1.

In another embodiment, one of the first and the second monoclonal antibody, or the first and second antigen-binding fragment thereof, has specificity for a peptide consisting of a first amino acid sequence from the C-terminal region of TK1 and the other of the first and the second monoclonal antibody, or the first and second antigen-binding fragment thereof, has specificity for a peptide consisting of the first amino acid sequence from the C-terminal region of TK1 or a second, different amino acid sequence from the C-terminal region of TK1.

In a further embodiment, one of the first and the second monoclonal antibody, or the first and second antigen-binding fragment thereof, has specificity for a peptide consisting of an amino acid sequence from the C-terminal region of TK1 and the other of the first and the second monoclonal antibody, or the first and second antigen-binding fragment thereof, has specificity for a conformation dependent epitope of human TK1.

In yet another embodiment, one of the first and the second monoclonal antibody, or the first and second antigen-binding fragment thereof, has specificity for a peptide consisting of an amino acid sequence from the active site of TK1 and the other of the first and second monoclonal antibody, or the first and second antigen-binding fragment thereof, has specificity for a conformation dependent epitope of human TK1.

An antigen-binding fragment of an antibody as used herein can be selected from a group consisting of a single chain antibody, a Fv fragment, a scFv fragment, a Fab fragment, a F(ab′)₂ fragment, a Fab′ fragment, a Fd fragment, a single-domain antibody (sdAb), a scFv-Fc fragment, a di-scFv fragment and a CDR region.

In an embodiment, the kit is a sandwich assay kit. In a particular embodiment, the kit is an enzyme-linked immunosorbent assay (ELISA) kit and preferably a sandwich ELISA.

In the discussion below, the first monoclonal antibody, or first antigen-binding fragment thereof, is assumed to be the capture antibody with the second monoclonal antibody, or second antigen-binding fragment thereof, acting as detection antibody. The embodiments are, however, not limited thereto but could switch capture and detection antibodies.

A sandwich ELISA can be used to detect STK1 material in a serum or plasma sample by preparing a surface of a support, such as a solid support, to which the first monoclonal antibody, or the first antigen-binding fragment thereof, is bound as so-called capture antibody. In a preferred embodiment, a known quantity of the first monoclonal antibody, or the first antigen-binding fragment thereof, is bound to the surface of the support. Any non-specific binding sites on the surface are optionally, but preferably, blocked. The serum or plasma sample is then applied to the surface so that any STK1 material present therein will be captured by the immobilized first monoclonal antibodies, or first antigen-binding fragments thereof. Unbound material is preferably removed by one or multiple washing steps. The second monoclonal antibody, or second antigen-binding fragment thereof, typically denoted detection antibody, is then added and is allowed to bind to any STK1 material captured by the first monoclonal antibody, or the first antigen-binding fragment thereof.

The amount of bound second monoclonal antibody, or second antigen-binding fragment thereof, is then determined by direct or indirect detection methods. For instance, a label or enzyme can be attached directly to the second monoclonal antibody, or the second antigen-binding fragment thereof, or indirectly via a link, such as a biotin-streptavidin or a biotin-avidin link. It is, alternatively, possible to use a secondary antibody, or secondary antigen-binding fragment thereof, that is labeled or connected to an enzyme and binds specifically to the second monoclonal antibody, or second antigen-binding fragment thereof.

Hence, in an embodiment, the second monoclonal antibody, or second antigen-binding fragment thereof, has a covalently attached biotin. Alternatively, the second monoclonal antibody, or second antigen-binding fragment thereof, has a covalently attached streptavidin or avidin.

The kit preferably also comprises a horseradish peroxidase (HRP) labeled streptavidin or a HRP labeled avidin. Alternatively, the kit also comprises a HRP labeled biotin. The kit also comprises a HRP substrate, such as a 3,3′,5,5′-tetramethylbenzidine (TMB) substrate, a 3,3′-diaminobenzidine (DAB) substrate or a 2,2′-azino-bis(3-ethylbenzothiazoline-6-sulphonic acid) (ABTS) substrate. In such a case, the level of STK1 material in the sample can be determined by spectrophotometric methods that detect the conversion of the chromogenic substrate by HRP into a colored product that is detectable.

In an embodiment, the kit also comprises a microtiter plate (MCP) as the support to which the first monoclonal antibody, or the first antigen-binding fragment thereof, is immobilized or is intended to be immobilized.

In an embodiment, one of the first monoclonal antibody, or the first antigen-binding fragment thereof, and the second monoclonal antibody, or the second antigen-binding fragment thereof, has specificity for an epitope consisting of GEAVAARKLF (SEQ ID NO: 1) and has a VH domain CDR1 having amino acid sequence DYEMH (SEQ ID NO: 5), a VH domain CDR2 having amino acid sequence AIHPGYGGTAYNQKFKG (SEQ ID NO: 6), a VH domain CDR3 having amino acid sequence FITKFDY (SEQ ID NO: 7), a VL domain CDR1 having amino acid sequence KSSQSLLDSDGKTFLN (SEQ ID NO: 8), a VL domain CDR2 having amino acid sequence LVSKLDS (SEQ ID NO: 9) and a VL domain CDR3 having amino acid sequence WQGTHFPWT (SEQ ID NO: 10).

In a particular embodiment, the other of the first monoclonal antibody, or the first antigen-binding fragment thereof, and the second monoclonal antibody, or the second antigen-binding fragment thereof, has specificity for the epitopes NCPVPGKPGE (SEQ ID NO: 2), PVPGKPGEAV (SEQ ID NO: 3) and NCPVPGKPGEAV (SEQ ID NO: 4) and has a VH domain CDR1 having amino acid sequence DYEMH (SEQ ID NO: 5), a VH domain CDR2 having amino acid sequence AILPGSGGTAYNQKFKG (SEQ ID NO: 11), a VH domain CDR3 having amino acid sequence LITTFDY (SEQ ID NO: 12), a VL domain CDR1 having amino acid sequence KSSQSLLDSDGKTYLN (SEQ ID NO: 13), a VL domain CDR2 having amino acid sequence LVSKLDS (SEQ ID NO: 9), and a VL domain CDR3 having amino acid sequence WQGTHFPWT (SEQ ID NO: 10).

An example of a TK1 ELISA that could be used according to the embodiments is AroCell TK 210 ELISA. The kit does not necessarily have to be an ELISA kit. In another embodiment, the kit uses affinity chromatography where the first monoclonal antibody, or the first antigen-binding fragment thereof, is bound to the stationary phase, such as to a gel matrix or beads in a column. For instance, the gel matrix or beads could be made of agarose, such as SEPHAROSER. In such a case, STK1 material present in a serum or plasma sample will be entrapped in the column through binding to the immobilized first monoclonal antibodies, or first antigen-binding fragments thereof. Following washing, the bound STK1 material can be eluted and detected using the second monoclonal antibody, or the second antigen-binding fragment thereof. For instance, the amount of eluted STK1 material can be determined using Western blotting and with the second monoclonal antibody, or the second antigen-binding fragment thereof, for STK1 detection using direct or indirect detection methods.

The support could alternatively be magnetic beads, such as DYNABEADS® magnetic beads.

In a further embodiment, the kit is a chemiluminescence immunoassay (CLIA) kit. CLIA is an immunoassay technique where the label is a luminescent molecule. CLIA methods can be direct, using luminophore markers, or indirect, using enzyme markers. Either method may be competitive or non-competitive. In direct CLIA methods, the luminophore markers used are typically acridinium and ruthenium esters, while the enzymatic markers used in indirect methods are typically alkaline phosphatase with adamantyl 1,2-dioxetane aryl phosphate (AMPPD) substrate and HRP with luminol or its derivatives as substrate.

Furthermore, the kit does not necessarily have to comprise a so-called capture antibody, or an antigen-binding fragment thereof. In clear contrast, multiple, i.e., at least two, different monoclonal antibodies, or antigen-binding fragments thereof, could be used to determine the level of STK1 material without the need for immobilizing at least one of the monoclonal antibodies, or the antigen-binding fragments thereof.

Carcinoma antigen 125 (CA 125), also referred to as ovarian cancer-related tumor marker CA 125 and mucin-16 (MUC-16), is a protein that in humans is encoded by the MUC16 gene. CA 125 is a member of the mucin family glycoproteins and has found application as a tumor marker or biomarker that may be elevated in the blood of some patients with specific types of cancers, most notably ovarian cancer, or other conditions that are benign.

Human epididymis protein 4 (HE4), also referred to as WAP four-disulfide core domain protein 2 (WFDC2), is a protein that in humans is encoded by the WFDC2 gene. HE4 is a tumor marker of ovarian cancer.

In an embodiment, determining the amount of CA 125 or HE4 comprises determining the amount of CA 125 or HE4 in the serum or plasma sample or another serum or plasma sample from the female human subject using an electrochemiluminescence immunoassay (ECLIA) kit. ECLIA is a quantitative method for measurement of antigen or antibody based on the change in electrochemiluminescence (ECL) signal before and after immunoreaction.

For instance, the amount of CA 125 in a serum or plasma sample could be determined or measured using Elecsys® CA 125 II by Roche and the amount of HE4 in a serum or plasma sample could be determined or measured using Elecsys® HE4 by Roche using, for instance, a Cobas® E411 analyzer.

Alternatively, the amount of CA 125 in a serum or plasma sample could be determined or measured using a human CA 125 ELISA kit, which are commercially available from different vendors, such as Abcam, RayBiotech, R&D Systems, Thermo Fisher Scientific, Abnova, etc. Correspondingly, the amount of HE4 in a serum or plasma sample could be determined or measured using a HE4 ELISA kit, which are commercially available from different vendors, such as Abcam, Thermo Fisher Scientific, Elabscience, R&D Systems, etc.

In an embodiment, the method also comprises selecting an anti-cancer treatment for the female human subject based on the predicted ovarian cancer risk. Thus, an optimal or at least suitable anti-cancer treatment is selected for the female human subject based on the determined amount of STK1 material and the determined amount of CA 125 or HE4 in the serum or plasma sample(s) and thereby based on the predicted ovarian cancer risk estimated for the female human subject. This means that female human subjects with a predicted high risk of ovarian cancer could be selected for a more aggressive anti-cancer treatment as compared to patients with a predicted lower risk of ovarian cancer. Examples of anti-cancer treatments that can be selected include one or more of surgery (hysterectomy, BSO) and chemotherapy. For instance, female human subjects predicted to have a high ovarian cancer risk could be selected for a first anti-cancer treatment, whereas other female human subjects with a lower ovarian cancer risk are selected for a second, different anti-cancer treatment.

In an embodiment, the method comprises selecting a patient surveillance schedule for the female human subject based on the predicted ovarian cancer risk for the female human subject. Thus, an optimal or at least suitable patient surveillance schedule or scheme is selected for the female human based on the ovarian cancer risk predicted for the female human subject. This means that female human subjects with a predicted high ovarian cancer risk could be selected for a more frequent surveillance and follow-up (first surveillance schedule) as compared to female human subjects with a predicted lower ovarian cancer risk, which instead can follow a less frequent surveillance and follow-up (second surveillance schedule).

### Example

Ovarian cancer is one of the most difficult tumors to detect and manage. Usually, it is diagnosed in late stage of the disease, which is associated with poor prognosis. Therefore, it is important to detect this cancer in early stages to improve overall survival. In this Example, TK1 protein and TK1 activity levels as well as the biomarkers CA 125, HE4, and ROMA index were determined. Elevated TK1 protein levels were found in both benign and malignant ovarian cancer patients. The combination of TK1 protein with CA 125 or HE4 showed higher sensitivity compared to the ROMA index. Therefore, the TK1 protein is a promising serum biomarker that can complement CA 125 or HE4 in the diagnostics of early stages of ovarian cancer.

### Materials and Methods

### Study Population and Sample Collection

This study included 134 serum samples from patients with ovarian tumors (72 had benign tumors and 62 had malignant ovarian cancer), and serum samples from 65 healthy women, used as the control group. The serum samples from healthy women and from women with ovarian tumors who attended the Department of Obstetrics and Gynecology, University Medical Centre Ljubljana, were obtained between April 2018 and May 2021. The blood samples were collected from all the patients before surgery and additional information was obtained regarding their lifestyle and gynecological and clinical status. For sample collection, strict standard operating procedures were followed, and serum was aliquoted and stored at −80° C. until analysis. The study was approved by the National Medical Ethics Committee of the Republic of Slovenia (Nr. 1Sep. 2, 2013). All patients gave their written consent for the diagnostic procedures, and surgery as well as inclusion in the study. The data of patients were collected as a prospectively designed database.

### Measurement of CA 125 and HE4

The serum CA 125 and HE4 levels were determined using in vitro quantitative fully automatic electrochemiluminescent immunoassays (ECLIAs) Elecsys® CA 125 II and Elecsys® HE4 on a Cobas® E411 immunoassay analyzer (Roche Diagnostics GmbH, Manheim, Germany). The method is based on the electrochemiluminescence immunoassay (ECLIA) principle, incorporating a sandwich immunoassay test principle. Serum HE4 and CA125 reference range were <140 μmol/L and <35 kU/L, respectively [8].

### The Risk of Ovarian Malignancy Algorithm (ROMA Index)

The premenopausal calculation formula of the ROMA index is as follows: PI=−12.0+(2.38×LN [HE4])+ (0.0626×LN [CA 125]) and the calculation for the postmenopausal was: PI=−8.09+(1.04×LN [HE4])+ (0.732×LN [CA 125]), wherein LN=Natural Logarithm. Higher risk of ovarian cancer for the premenopausal women was with a ROME value ≥11.4% and postmenopausal women had a higher risk with a ROMA value ≥29.9% as described previously [8].

### Serum TK Activity (STK1a) and TK1 Protein (STK1p) Determinations

The TK activity in all the serum samples was analyzed by the LIAISON® assay as described previously and the TK activity is expressed in U/L [10]. The TK1 protein levels in serum samples was measured by using AroCell TK 210 ELISA. The AroCell TK 210 ELISA is a sandwich ELISA that utilizes two monoclonal anti-TK antibodies against the C-terminal region of human TK1. The assay was performed according to the manufacturer's instructions as previously described and STK1p levels were expressed in ng/ml [11].

### Statistical Analysis

The levels of biomarkers including CA 125, HE4, STK1a, and STK1p levels in healthy, benign, and malignant ovarian cancer serum samples were evaluated for normality using the D'Agostino and Pearson omnibus normality test. For continuous variables, the significance of differences was tested by the Mann-Whitney U test or Wilcoxon signed-rank test according to the comparison of independent samples or paired samples. Logistic regression analysis was performed to establish the best possible combination of the biomarkers. The diagnostic performance of all possible combinations were evaluated by the receiver operating characteristic (ROC) curves and the area under the curve (AUC) with 95% confidence intervals (95% CIs). All statistical analyses were performed using GraphPad Prism 8.0 (GraphPad Software, La Jolla, CA, USA) and MedCalc 17. 6. Statistical significance was achieved when P<0.05.

### Results

### Patient Characteristics

During the period of 2018 to 2021, a total of 199 (134 patients with ovarian tumors and 65 healthy controls) were included in this study. Among the 134 included women with ovarian tumors, 72 had benign tumors and 62 had malignant ovarian cancer. Serous tumors were the most common carcinomas (58%) followed by mucinous tumors (15%) and endometrial tumors (15%).

The mean±SD of age in years was 57.0±3.7 (range=26.9-85.7) in all patients. Twenty-one patients were in premenopausal status in the range of 26.9-50.2 years (mean±SD=40.5±3.3) while 41 patients were in the postmenopausal status of 52.5-85.7 years (mean±SD=65.5±2.9). In the benign ovarian disease group 17% were ovarian endometriosis, 15% serous cystadenoma, followed by 13% serous cystadenofibroma and 10% of ovarian mucinous cystadenoma. The patients ages were 14.9-85.8 years, with a mean±SD of 52.01±0.3.8 years. Of the 72 benign tumor patients, 33 patients were in premenopausal status and their ages were 37.6±3.4 while for the 39 patients in postmenopausal status it was 54.3±2.9 years. Of the 65 healthy controls, 43 cases were at a premenopausal status with the mean±SD of age was of 42.6±2.5 years and for the 21 cases of postmenopausal status it was 55.1±2.4 years. All the patient characteristics are summarized in Table 1.

TABLE 1
Histological types and distribution of stages of diseases for all patients as well as the distribution
of the premenopausal and postmenopausal women.

| | all patients (n) | menopausal status | |
|---|---|---|---|
| | 134 | pre- (n) | post- (n) |
| benign ovarian diseases (histologic type) | 72 | 33 | 39 |
| serous cystadenoma | 11 | 2 | 9 |
| serous cystadenofibroma | 9 | 1 | 8 |
| mucinous cystadenoma | 7 | 4 | 3 |
| mucionous cystadenofibroma | 2 | 0 | 2 |
| Endometriosis | 12 | 12 | 0 |
| benign Brenner tumor | 1 | 0 | 1 |
| sclerozing stromal tumor | 1 | 1 | 0 |
| mature teratoma | 8 | 5 | 3 |
| follicle cyst | 2 | 2 | 0 |
| corpus luteum cyst | 1 | 1 | 0 |
| inclusion cyst | 7 | 2 | 5 |
| simple cyst | 9 | 3 | 6 |
| cellular fibroma | 2 | 0 | 2 |
| malign ovarian disease (histologic type) | 62 | 21 | 41 |
| serous borderline tumor | 9 | 6 | 3 |
| Low-grade serous carcinoma | 4 | 1 | 3 |
| High-grade serous carcinoma | 17 | 3 | 14 |
| mucinous borderline tumor | 7 | 5 | 2 |
| mucinous carcinoma | 2 | 1 | 1 |
| endometrioid carcinoma | 8 | 2 | 6 |
| endometrioid borderline tumor | 1 | 0 | 1 |
| clear cell carcinoma | 2 | 1 | 1 |
| seromucinous borderline tumor | 3 | 0 | 3 |
| adult granulosa cell tumor | 2 | 1 | 1 |
| Dysgerminoma | 1 | 1 | 0 |
| High-grade primary peritoneal serous carcinoma | 5 | 0 | 5 |
| Low-grade primary peritoneal serous carcinoma | 1 | 0 | 1 |
| Healthy controls | 65 | 43 | 22 |
| FIGO stage | 62 | | |
| I | 29 | 13 | 16 |
| II | 5 | 2 | 3 |
| III | 24 | 6 | 18 |
| IV | 4 | 0 | 4 |
| Grade | 62 | | |
| G1 | 35 | 16 | 19 |
| G2 | 6 | 3 | 3 |
| G3 | 21 | 2 | 19 |

### TK1 Levels in the Different Patient Groups

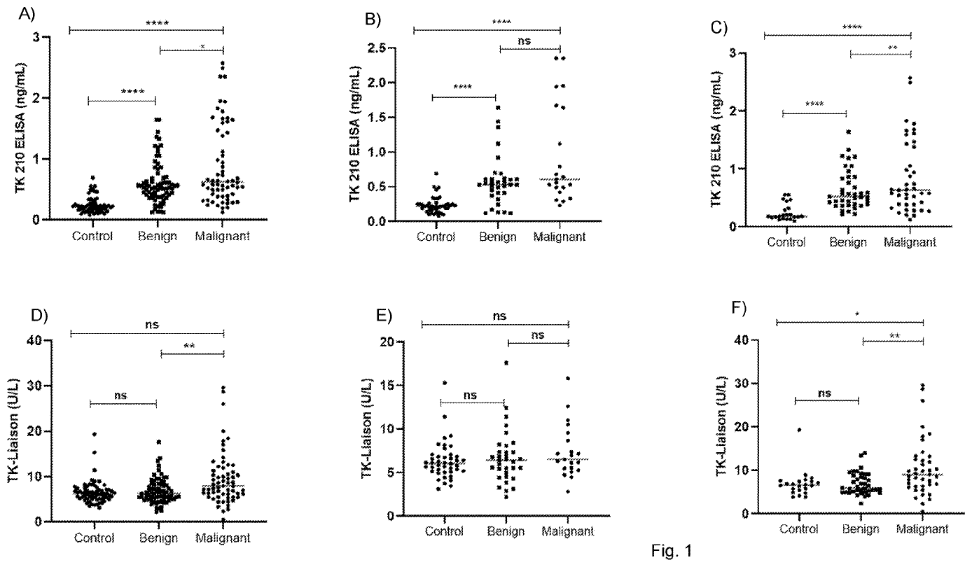

Both serum TK1 protein levels (STK1p) and TK1 activity levels (STK1a) were determined and compared with healthy controls (n=65), benign (n=72) and malignant (n=62) ovarian cancer patients using the AroCell TK 210 ELISA and the LIAISON® assay. STK1p concentrations were significantly higher in the malignant ovarian cancer group compared to the benign and the healthy control groups (P<0.0001, FIG. 1A). These results demonstrate that malignant ovarian cancer patients have higher levels of STK1p than patients in the benign and healthy control groups. A comparison of STK1p between malignant and benign showed a significant difference between the pre-versus postmenopausal women (FIGS. 1B and 1C). For postmenopausal women, STK1p was significantly higher in the malignant compared to benign groups, while there was no significant difference between the malignant and the benign cancer groups in case of premenopausal women (P=0.34). The STK1a values showed another type of results with no significant differences between the control group and the malignant ovarian cancer or the benign ovarian cancer groups (FIG. 1D). However, the malignant ovarian tumor group had significantly higher STK1a levels compared to the group of benign cancer patients. Furthermore, no significant differences were found in the STK1a levels in these groups in premenopausal women (FIG. 1E). However, significant differences in the STK1a levels were observed between malignant and benign as well as healthy control groups in postmenopausal women (FIG. 1F).

In all these groups, CA 125 and HE4 values were determined and the ROMA index values were also calculated as described in materials and methods. The CA 125 levels were significantly higher in the malignant ovarian cancer group compared to the benign group and healthy controls. Furthermore, the CA 125 levels in the malignant group were significantly higher than those in the benign ovarian cancer group. However, both the HE4 and ROMA index values were significantly higher in the malignant ovarian cancer compared to healthy control groups but there was no difference between benign and healthy control groups.

There was no significant difference in STK1p levels between premenopausal and postmenopausal women in the healthy controls, nor in case of the benign and malignant ovarian cancer groups (Table 2). Similar results were observed with STK1a, CA 125, and HE4 in the healthy control groups. The levels of CA 125 in premenopausal women were significantly higher compared to postmenopausal in the benign cancer groups. In contrast, the HE4 levels in postmenopausal women were significantly higher than premenopausal in the begin cancer group (Table 2). In the case of malignant ovarian tumor group, the STK1a, CA 125 and HE4 levels were significantly higher for postmenopausal than for premenopausal women.

TABLE 2
STK1p, STK1a, CA 125, and HE4 levels in different groups.

| | n | STK1p (ng/ml) | STK1a (U/L) | CA 125 (kU/L) | HE4 (nmol/L) |
|---|---|---|---|---|---|
| Healthy controll | | | | | |
| All | 64 | 0.21 | 6.2 | 12.2 | 47.2 |
| | | (0.16-0.27) | (5.1-7.1) | (9.8-17.7) | (42.1-54-9) |
| Premenopausal | 42 | 0.22 | 6.1 | 13.2 | 45.4 |
| | | (0.17-0.25) | (5.1-6.9) | (10.3-19.8) | (38.2-56.9) |
| Postmenopausal | 22 | 0.18 | 6.6 | 10.7 | 49.7 |
| | | (0.16-0.35) | (5.3-7.5) | (8.8-16.1) | (44.5-53.5) |
| Benign masses | | | | | |
| All | 72 | 0.53 | 6.3 | 17.5 | 50.0 |
| | | (0.39-0.70) | (5.0-8.2) | (10.5-41.2) | (42.0-66.4) |
| Premenopausal | 33 | 0.53 | 6.4 | 31.9 | 46.8 |
| | | (0.39-0.62) | (4.7-7.7) | (15.2-69.1) | (39.3-59.0) |
| Postmenopausal | 39 | 0.52 | 5.0 | 13.9 | 57.8 |
| | | (0.39-0.86) | (4.9-8.8) | (9.0-25.2) | (44.7-75.6) |
| Malignant ovarian tumors | | | | | |
| All | 62 | 0.62 | 7.9 | 103.8 | 136.5 |
| | | (0.38-1.44) | (6.0-11.4) | (21.4-444) | (62.4-417.8) |
| Premenopausal | 21 | 0.61 | 6.5 | 42.7 | 52.0 |
| | | (0.45-1.65) | (5.4-9.1) | (19.3-193) | (40.5-128.6) |
| Postmenopausal | 41 | 0.63 | 9.0 | 169.2 | 290.8 |
| | | (0.38-1.40) | (6.3-12.8) | (27.6-700) | (93-972) |

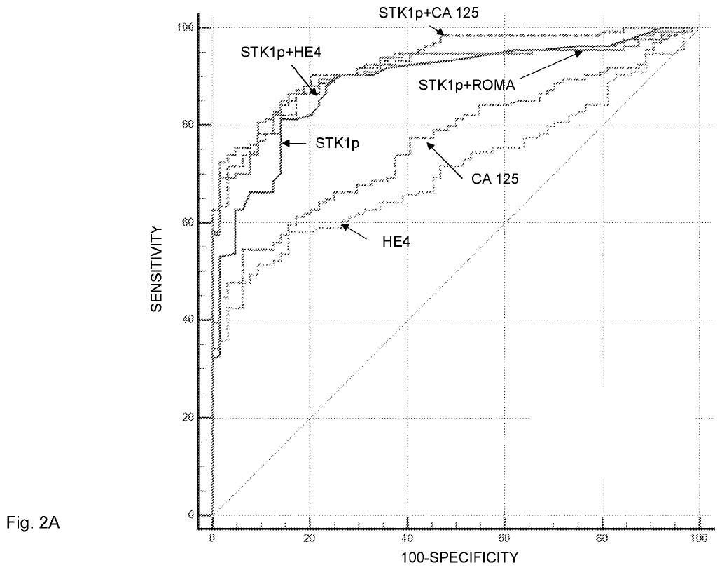

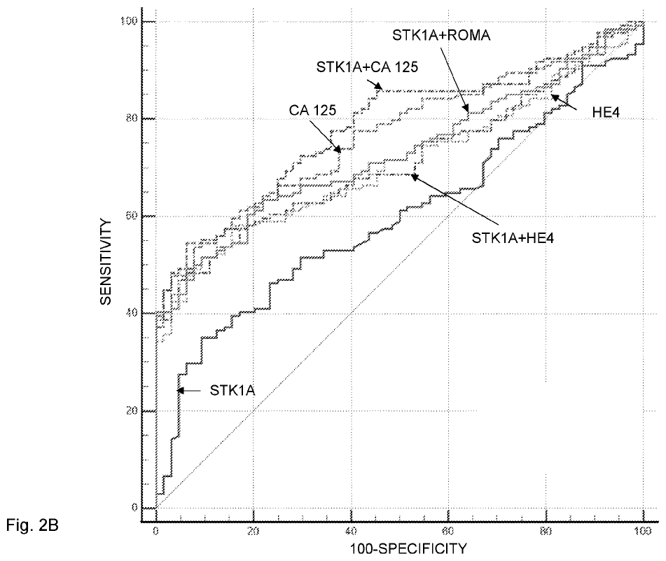

The results of univariate and multivariate receiver operating characteristic (ROC) curve analysis are shown in Table 3. The ROC area under curves (AUCs) and standard errors were based on the complete data set from 134 ovarian tumor serum samples and 64 healthy controls using STK1p, CA 125, HE4, 5 STK1p+CA 125, STK1p+HE4 and STK1p+ROMA index biomarkers. Multivariate ROC analyses of the combinations of the STK1p, STK1p+CA 125, STK1p+HE4 and STK1p+ROMA index biomarkers were performed (FIGS. 2A and 2B). Among all the possible combinations, three dual combinations had ROC AUC above 0.90. These three combinations were further evaluated for their performance at distinguishing ovarian cancer samples from healthy controls.

TABLE 3
Univariate and multivariate ROC analysis of biomarkers.

| Marker name | ROC AUC ± SE (AUC) |
|---|---|
| STK1p | 0.88 ± 0.02 |
| STK1a | 0.60 ± 0.04 |
| CA 125 | 0.77 ± 0.03 |
| HE4 | 0.71 ± 0.04 |
| ROMA Index | 0.73 ± 0.03 |
| STK1p + STK1a | 0.89 ± 0.02 |
| STK1p + CA 125 | 0.93 ± 0.02 |
| STK1p + HE4 | 0.88 ± 0.02 |
| STK1p + CA 125 + HE4 | 0.94 ± 0.02 |
| STK1p + ROMA Index | 0.91 ± 0.02 |
| STK1a + CA 125 | 0.78 ± 0.03 |
| STK1a + HE4 | 0.71 ± 0.04 |
| STK1a + CA 125 + HE4 | 0.80 ± 0.03 |
| STK1a + ROMA Index | 0.73 ± 0.03 |

### All Ovarian Cancers Versus all Healthy Controls

FIG. 2A shows the ROC curves for STK1p alone, CA 125 alone, HE4 alone, and the dual markers (STK1p+CA 125, STK1p+HE4 and STK1p+ROMA Index) when these combinations were calculated for the 198 samples (134 ovarian tumor and 64 healthy controls). Each biomarker and combination was evaluated for sensitivity, specificity, positive predictive value (PPV), and negative predictive value (NPV) at the ROC curves cut point with a specificity of around 95%.

Table 4 shows the results of STK1p in combination with CA 125, HE4, and ROMA Index attained a sensitivity above 70% with a specificity of 95%.

TABLE 4
ROC curve analysis for the different biomarkers alone, and in combinations.

| Biomarker | Cut-off | AUC | Sensitivity | Specificity | PPV | NPV |
|---|---|---|---|---|---|---|
| STK1p | 0.50 | 0.88 | 62.9% | 95.3% | 96.6% | 55.1% |
| STK1a | 9.10 | 0.60 | 27.6% | 95.3% | 90.2% | 38.2% |
| CA 125 | 26.4 | 0.77 | 54.5% | 93.8% | 94.8% | 49.6% |
| HE4 | 65.5 | 0.71 | 47.8% | 93.8% | 94.1% | 46.2% |
| ROMA Index | 16.7 | 0.73 | 42.5% | 95.3% | 95.0% | 44.2% |
| STK1p + CA 125 | 0.78 | 0.93 | 75.3% | 95.3% | 97.1% | 64.9% |
| STK1p + HE4 | 0.80 | 0.91 | 73.8% | 93.8% | 96.1% | 63.2% |
| STK1p + ROMA Index | 0.83 | 0.91 | 70.2% | 95.3% | 96.9% | 60.4% |
| STK1a + CA 125 | 0.76 | 0.78 | 49.3% | 95.3% | 95.7% | 47.3% |
| STK1a + HE4 | 0.72 | 0.71 | 47.1% | 95.3% | 95.5% | 46.2% |
| STK1a + ROMA Index | 0.73 | 0.73 | 44.1% | 95.3% | 95.2% | 44.9% |

FIG. 2B shows the ROC curves for STK1a alone, CA 125 alone, and HE4 alone and the dual markers (STK1a+CA 125, STK1a+HE4, and STK1a+ROMA Index). Overall, STK1a and the combination of STK1a with other assays showed lower sensitivity compared to STK1p. Furthermore, the combination of CA 125 with STK1p showed higher sensitivity and PPV as compared to alone (Table 4).

Based on these results, further analysis was carried out using STK1p, STK1p+CA 125, STK1p+HE4, and STK1p+ROMA index. The patient sera sub-grouped as all premenopausal, all postmenopausal women, benign and malignant cancers. The assay performances were evaluated for these subgroups by ROC curve analysis as shown in Table 5.

TABLE 5
ROC curve analysis of STK1p in combination with other biomarkers in different subgroups.

| Premenopausal vs controls | Cut-off | AUC | Sensitivity | Specificity | PPV | NPV |
|---|---|---|---|---|---|---|
| All stages | | | | | | |
| STK1p | 0.49 | 0.88 | 64.8% | 95.2% | 94.6% | 67.8% |
| STK1p + CA 125 | 0.68 | 0.95 | 81.5% | 95.2% | 95.7% | 80.0% |
| STK1p + HE4 | 0.75 | 0.88 | 72.2% | 95.2% | 95.1% | 72.7% |
| STK1p + ROMA Index | 0.75 | 0.88 | 72.2% | 95.2% | 95.1% | 72.7% |
| Postmenopausal vs controls | | | | | | |
| All stages | | | | | | |
| STK1p | 0.55 | 0.89 | 55.0% | 100% | 100% | 37.9% |
| STK1p + CA 125 | 0.86 | 0.92 | 73.8% | 100% | 100% | 51.2% |
| STK1p + HE4 | 0.86 | 0.93 | 72.5% | 100% | 100% | 50.0% |
| STK1p + ROMA Index | 0.83 | 0.94 | 78.8% | 100% | 100% | 56.4% |
| Benign vs Controls | | | | | | |
| STK1p | 0.50 | 0.87 | 59.7% | 95.3% | 93.5% | 68.1% |
| STK1p + CA 125 | 0.76 | 0.91 | 63.9% | 96.9% | 95.9% | 71.3% |
| STK1p + HE4 | 0.77 | 0.88 | 61.1% | 95.3% | 93.6% | 68.5% |
| STK1p + ROMA Index | 0.79 | 0.88 | 59.7% | 95.3% | 93.5% | 67.8% |
| Malignant vs Controls | | | | | | |
| STK1p | 0.50 | 0.90 | 66.1% | 95.3% | 93.2% | 74.7% |
| STK1p + CA 125 | 0.65 | 0.95 | 80.7% | 100% | 100% | 84.2% |
| STK1p + HE4 | 0.577 | 0.95 | 80.7% | 100% | 100% | 84.2% |
| STK1p + ROMA Index | 0.606 | 0.95 | 82.3% | 100% | 100% | 85.1% |
| Malignant vs Benign | | | | | | |
| STK1p | 1.36 | 0.61 | 27.5% | 95.8% | 85% | 60.5% |
| STK1p + CA 125 | 0.56 | 0.73 | 43.5% | 95.8% | 90% | 66.3% |
| STK1p + HE4 | 0.60 | 0.82 | 58.1% | 97.2% | 94.7% | 72.9% |
| STK1p + ROMA Index | 0.59 | 0.82 | 66.1% | 95.8% | 93.2% | 76.7% |

### Performance Evaluation of Dual Biomarkers in Sub-Groups of Ovarian Cancer Patients

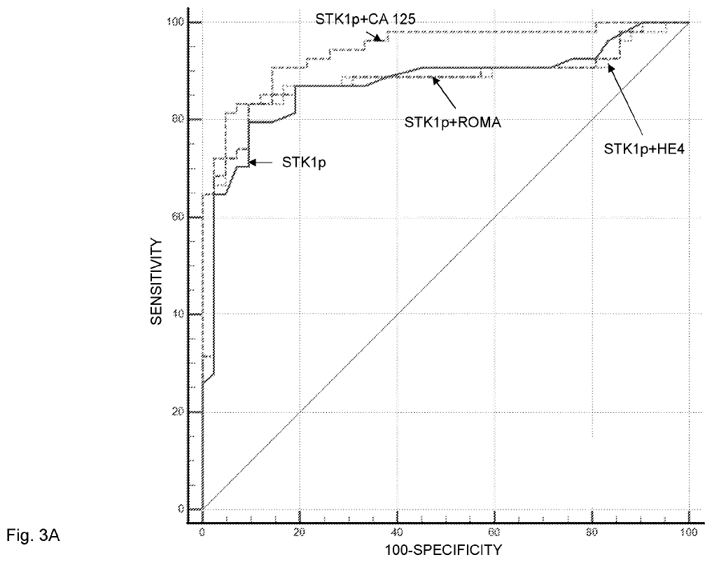

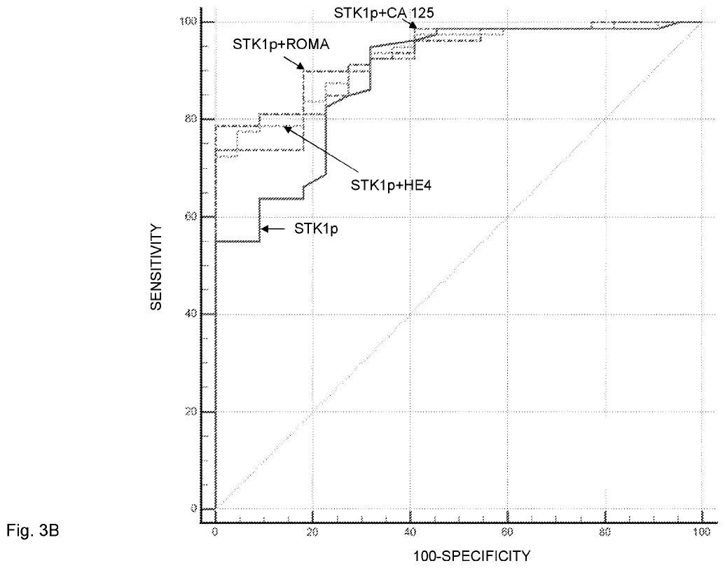

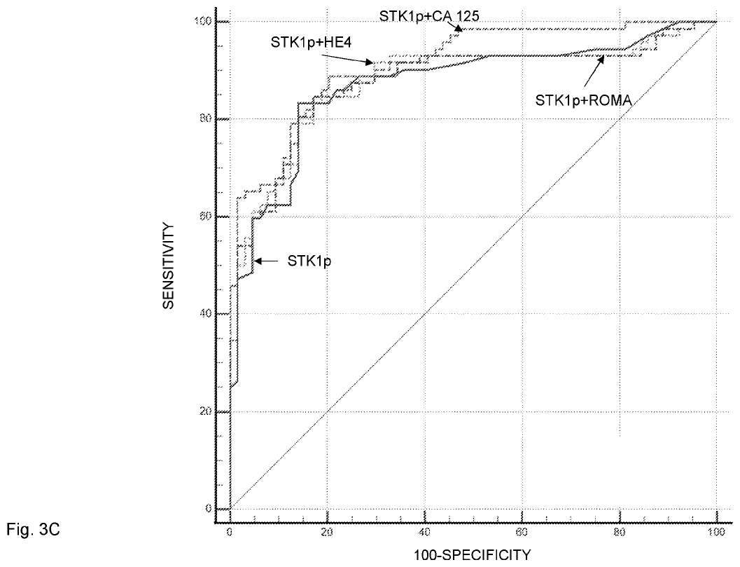

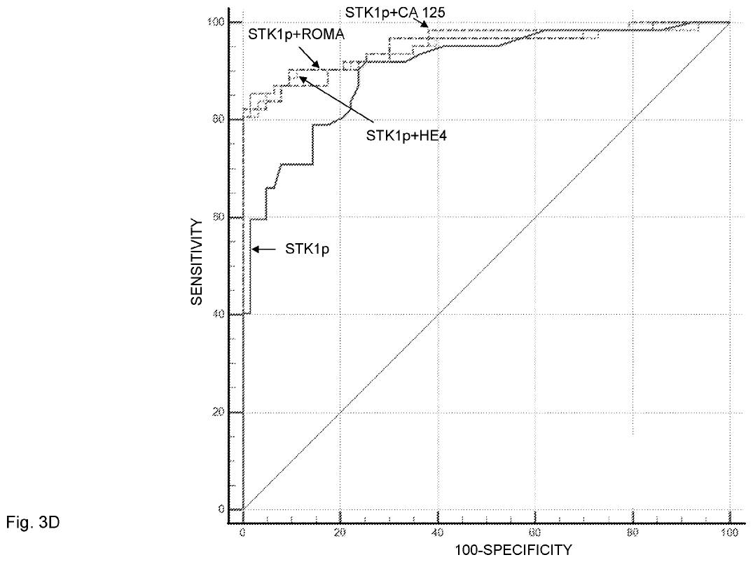

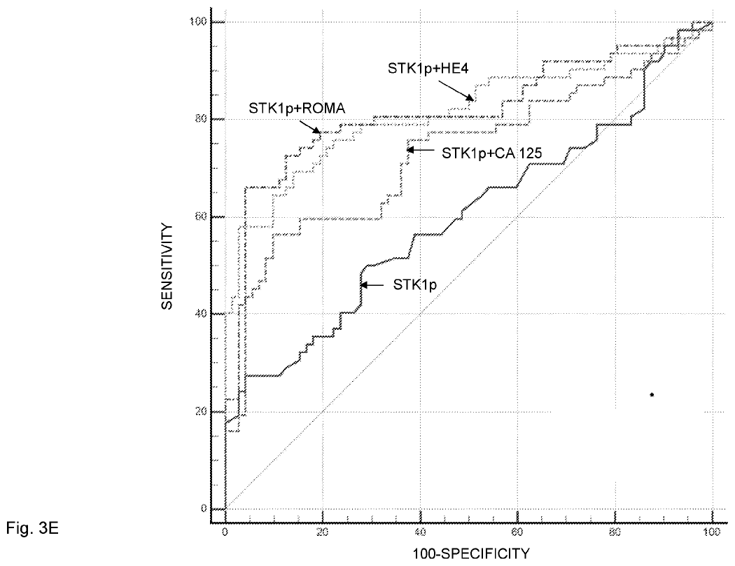

The performance of combination of biomarkers with STK1p was evaluated in subgroups of the ovarian cancer patients by using ROC curve analysis. The diagnostic performances of STK1p alone, STK1p+CA 125, STK1p+HE4, and STK1p+ROMA index are shown in FIG. 3 and Table 5. In the detection of all stages of cancers, the AUC for STK1p+CA 125 was 0.945 (95% CI: 0.879-0.981) for premenopausal women (FIG. 3A), which is higher than the STK1p+ROMA index (AUC=0.877, 95% CI: 0.795-0.936), and the AUC of STK1p+ROMA index (AUC=0.936, 95% CI=0.87-0.97) was higher than that of STK1p+CA 125 (AUC=0.920, 95% CI=0.85-0.965) for postmenopausal women (FIG. 3B). In the detection of benign mass from healthy controls, the AUC for STK1p+CA 125 (AUC=0.914, 95% CI=0.85-0.95) was higher than STK1p+HE4 (AUC=0.877, 95% CI=0.81-0.927) and STK1p+ROMA index (AUC=0.876, 95% CI=0.809-0.926) (FIG. 3C). For differentiation of malignant ovarian cancer from healthy controls, all three dual biomarkers i.e., STK1p+CA 125, STK1p+HE4, and STK1p+ROMA index showed higher AUCs compared to STK1p alone (Table 5) (FIG. 3D). In the differentiation of benign mass from malignant ovarian cancer, the AUC was significantly higher for the STK1p+ROMA index (AUC=0.819, 95% CI=0743-0.88) followed by STK1p+HE4 (AUC=0.817, 95% CI=0.741-0.878) and STK1p+CA 125 (AUC=0.731, 95% CI=0.648-0.804) compared to STK1p (AUC=0.60, 95% CI=0.52-0.685) alone (Table 5) (FIG. 3E). These results demonstrate that the combination of STK1p+CA 125 give the best diagnostic performance in the detection of early-stage ovarian cancer in comparison with the group with benign tumors.

For premenopausal women, STK1p+CA 125 showed higher PPV and NPV than the STK1p+ROMA index (95.7% vs 95.1% and 80.0% vs 72.7%). Whereas in the case of postmenopausal women, the PPV values were similar in all three combinations (100%) with a higher NPV for STK1p+ROMA index (56.4%) followed by STK1p+CA 125 (51.2%) and STK1p+HE4 (50.0%). These results strongly indicate that the dual biomarker STK1p+CA 125 had the best diagnostic performance for the detection of benign as well as malignant ovarian cancers in comparison to the healthy control group. However, in the differentiation of benign from malignant ovarian cancers the combination of STK1p+ROMA index gave higher sensitivity (66.1%) than the other combinations as shown in Table 5.

A similar analysis was performed by using STK1a determinations in these subgroups. In this case STK1a+CA 125 showed the highest sensitivity than compared to STK1a+HE4 and STK1a+ROMA index in differentiation of premenopausal women, benign as well as malignant ovarian tumors from healthy controls. For differentiation of postmenopausal women with cancer, STK1a+ROMA index showed higher sensitivity compared to the other combinations. These results are similar to the STK1p combination with CA 125 giving the highest sensitivity for early detection of ovarian cancer.

Based on these results, further analysis was carried out using STK1p+CA 125, STK1p+HE4, STK1a+CA 125, and STK1a+HE4 combinations. The malignant ovarian cancer sera were subclassified based on FIGO staging and the STK1p+CA 125 (P<0.0001, FIG. 4A) as well as STK1a+CA 125 (P=0.0104, FIG. 4B) in stage III+IV patients were significantly higher compared to stage I+II patients. Furthermore, ROC analysis showed that STK1p+CA 125 (AUC=0.81, sensitivity=50% with a specificity=92%) had higher capacity to differentiate stage I+II from stage III+IV than STK1a+CA 125 (AUC=0.69, sensitivity=35.7% with a specificity=92%) (FIG. 4C). Similar results were obtained with the combination of STK1p+HE4 and STK1a+HE4 (FIGS. 4D and 4E) and ROC curve analysis showed AUC of 0.82 for STK1p+HE4 and 0.80 for STK1a+HE4.

### STK1p and STK1a Levels Before and After Surgery

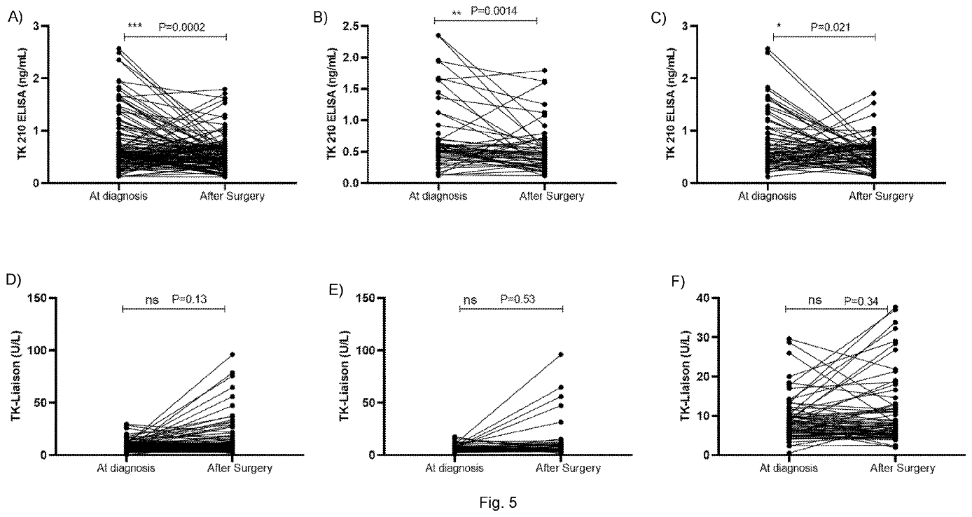

Both benign and malignant ovarian cancer patients (n=123) were followed after surgery, and the STK1p levels were significantly reduced after surgery for all (P=0.0002, FIG. 5A), pre-(P=0.0014, FIG. 5B) as well as in case of postmenopausal women (P=0.021, FIG. 5C). Among the 123 patients, a total of 81 patients, accounting for 61%, showed a downward trend for the STK1p levels when compared to the preoperative levels. In contrast, there was no significant difference in STK1a levels between preoperative and postoperative patients with benign as well as malignant cancer (FIGS. 5D, 5E, and 5F).

Furthermore, there was a significant correlation between the ratio of STK1p at diagnosis/STK1p after surgery and with the number of days after surgery (FIG. 6A) (rs=0.25, P=0.0072). In addition, the STK1p was measured in 23 malignant ovarian cancer patients after chemotherapy. With the exception of 4 patients, the remaining patients showed a significant decrease in STK1p levels after chemotherapy (P=0.013, FIG. 6B). During follow-up, 9 patients out of 32 had relapse of the disease. The patients with relapse after chemotherapy had significantly higher levels of STK1p+CA 125 as well as STK1p+HE4 compared to patients without relapse (FIGS. 6C and 6D). Similar results were observed with the STK1a determinations as shown in FIGS. 6E and 6F.

The results of this study showed that the combination of STK1p with CA 125 or HE4 could improve the sensitivity and specificity for early detection of ovarian cancer. The combination of these biomarker offers a route to improve detection of patients with ovarian cancer in the early stages of the disease leading to higher chance of curative treatment.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

### REFERENCES

1. Hellstrom, et al., The HE4 (WFDC2) protein is a biomarker for ovarian carcinoma, Cancer research, (2003) 63(13): 3695-3700.
2. Drapkin, et al., Human epididymis protein 4 (HE4) is a secreted glycoprotein that is overexpressed by serous and endometrioid ovarian carcinomas, Cancer research, (2005) 65(6): 2162-2169.
3. Hada et al., A, Comparison of the predictive performance of risk of malignancy indexes 1-4, HE4 and risk of malignancy algorithm in the triage of adnexal masses, Journal of ovarian research, (2020) 13(1): 1-9.
4. Li, et al., Does risk for ovarian malignancy algorithm excel human epididymis protein 4 and CA125 in predicting epithelial ovarian cancer: a meta-analysis, BMC Cancer, (2012) 12(1): 1-18.
5. Molina et al., HE4 a novel tumor marker for ovarian cancer: comparison with CA 125 and ROMA algorithm in patients with gynecological diseases, Tumor Biology, (2011) 32(6): 1087-1095.
6. Moore, et al., The use of multiple novel tumor biomarkers for the detection of ovarian carcinoma in patients with a pelvic mass, Gynecologic Oncology, (2008) 108(2): 402-408.
7. Jacob et al., No benefit from combining HE4 and CA125 as ovarian tumor markers in a clinical setting, Gynecologic oncology, (2011) 121(3): 487-491.
8. Zhu, et al., A combined strategy of TK1, HE4 and CA125 shows better diagnostic performance than risk of ovarian malignancy algorithm (ROMA) in ovarian carcinoma, Clinica Chimica Acta, (2022) 524:43-
50.
9. Jagarlamudi et al., Breast, and prostate cancer patients differ significantly in their serum thymidine kinase 1 (TK1) specific activities compared with those hematological malignancies and blood donors: implications of using serum TK1 as a biomarker, BMC Cancer (2015) 15:66.
10. Ohrvik et al., Sensitive nonradiometric method for determining thymidine kinase 1 activity, Clinical Chemistry, (2004) 50(9): 1597-1606.
11. Jagarlamudi et al., Analytical and clinical characterization of an optimized dual monoclonal sandwich ELISA for the quantification of thymidine kinase 1 (TK1) protein in human blood samples, PLOS One, (2022) 17(10): e0275444.
12. Xi et al., Research on application value of combined detection of serum CA125, HE4 and TK1 in the diagnosis of ovarian cancer, European Review for Medical and Pharmacological Sciences (2017) 21:4536-4541.

1-21. (canceled)

22. A method for predicting ovarian cancer risk for a female human subject comprising:
determining an amount of serum thymidine kinase 1 (STK1) material in a serum or plasma sample from the female human subject using a kit comprising a first monoclonal antibody, or a first antigen-binding fragment thereof, specifically binding to a serum form of human TK1 and a second monoclonal antibody, or a second antigen-binding fragment thereof, specifically binding to the serum form of human TK1;
determining an amount of carcinoma antigen 125 (CA 125) or human epididymis protein 4 (HE4) in the serum or plasma sample or another serum or plasma sample from the female human subject; and
predicting ovarian cancer risk for the female human subject based on the amount of STK1 material and the amount of one, but not both, of CA 125 and HE4.

23. The method according to claim 22, wherein the female human subject is in a premenopausal status.

24. The method according to claim 22, wherein predicting ovarian cancer risk comprises predicting an ovarian cancer stage for the female human subject based on the amount of STK1 material and the amount of one, but not both, of CA 125 and HE4.

25. The method according to claim 24, wherein predicting the ovarian cancer stage comprises predicting whether the female human subject is suffering from a FIGO stage I or II ovarian cancer or a FIGO stage III or IV stage ovarian cancer based on the amount of STK1 material and the amount of one, but not both, of CA 125 and HE4.

26. The method according to claim 22, wherein
determining the amount of STK1 material comprises determining, using the first monoclonal antibody, or the first antigen-binding fragment thereof, specifically binding to the serum form of human TK1, and the second monoclonal antibody, or the second antigen-binding fragment, thereof, specifically binding to the serum form of human TK1 the amount of STK1 material in a serum or plasma sample taken from the female human subject following treatment for ovarian cancer;
determining the amount of CA 125 or HE4 comprises determining the amount of CAR 125 or HE4 in the serum or plasma sample or another serum or plasma sample taken from the female human subject following treatment for ovarian cancer; and
predicting ovarian cancer risk comprises predicting relapse of ovarian cancer for the female human subject based on the amount of STK1 material and the amount of one, but not both, of CA 125 and HE4.

27. The method according to claim 22, wherein predicting ovarian cancer risk comprises predicting whether the female subject is suffering from ovarian cancer or does not suffer from ovarian cancer based on the amount of STK1 material and the amount of one, but not both, of CA 125 and HE4.

28. The method according to claim 27, wherein predicting ovarian cancer risk comprises predicting whether the female subject is suffering from benign ovarian cancer or does not suffer from ovarian cancer based on the amount of STK1 material and the amount of one, but not both, of CA 125 and HE4.

29. The method according to claim 27, wherein predicting ovarian cancer risk comprises predicting whether the female subject is suffering from malignant ovarian cancer or does not suffer from ovarian cancer based on the amount of STK1 material and the amount of one, but not both, of CA 125 and HE4.

30. The method according to claim 22, wherein
determining the amount of CA 125 or HE4 comprises determining an amount of CA 125 in the serum or plasma sample or the another serum or plasma sample from the female human subject; and
predicting ovarian cancer risk comprises predicting ovarian cancer risk for the female human subject based on the amount of STK1 material and the amount of CA 125 but not on amount of HE4.

31. The method according to claim 30, wherein predicting ovarian cancer risk comprises predicting ovarian cancer risk for the female human subject based solely on the amount of STK1 material and the amount of CA 125.

32. The method according to method 22, wherein determining the amount of STK1 material in the serum or plasma sample comprises:
contacting the serum or plasma sample with the first monoclonal antibody, or the first antigen-binding fragment thereof, and the second monoclonal antibody, or the second antigen-binding fragment thereof; and
measuring an amount of the first monoclonal antibody, or the first antigen-binding fragment thereof, or the second monoclonal antibody, or the second antigen-binding fragment bound to the STK1 material.

33. The method according to claim 32, further comprising correlating the measured amount of the first monoclonal antibody, or the first antigen-binding fragment thereof, or the second monoclonal antibody, or the second antigen-binding fragment thereof, bound to the STK1 material to an amount of STK1 material.

34. The method according to claim 33, wherein correlating the measured amount of the first monoclonal antibody, or the first antigen-binding fragment thereof, or the second monoclonal antibody, or the second antigen-binding fragment thereof, comprises correlating the measured amount of the first monoclonal antibody, or the first antigen-binding fragment thereof, or the second monoclonal antibody, or the second antigen-binding fragment thereof, to an amount of STK1 material using a pre-defined correlation between measured amount of the first monoclonal antibody, or the first antigen-binding fragment thereof, or the second monoclonal antibody, or the second antigen-binding fragment thereof, bound to recombinant human TK1 and a concentration of recombinant human TK1.

35. The method according to claim 22, further comprising adding a sample dilution buffer to the serum or plasma sample, wherein the sample dilution buffer comprises:
adenosine triphosphate in a concentration selected within an interval of from 0.5 mM up to 50 mM; and
a reducing agent selected from the group consisting of dithioerythritol (DTE), dithiothreitol (DTT), dithiobutylamin (DTBA), tris(2-carboxyethyl) phosphine) (TCEP), and any combination thereof, and in a concentration selected within an interval of from 0.1 mM up to 10 mM.

36. The method according to claim 22, wherein
one of the first monoclonal antibody, or the first antigen-binding fragment thereof, and the second monoclonal antibody, or the second antigen-binding fragment thereof, has specificity for a peptide consisting of an amino acid sequence from the C-terminal region of TK1; and
the other of the first monoclonal antibody, or the first antigen-binding fragment thereof, and the second monoclonal antibody, or the second antigen-binding fragment thereof, has specificity for a peptide selected from the group consisting of a peptide consisting of the amino acid sequence from the C-terminal region of TK1, a peptide consisting of another amino acid sequence from the C-terminal region of TK1 and a peptide consisting of an amino acid sequence from the active site of TK1.

37. The method according to claim 22, wherein
the first monoclonal antibody, or the first antigen-binding fragment thereof, is selected from the group consisting of:
a monoclonal antibody, or an antigen-binding fragment thereof, having specificity for GEAVAARKLF (SEQ ID NO: 1) of human TK1;
a monoclonal antibody, or an antigen-binding fragment thereof, having specificity for at least one of NCPVPGKPGE (SEQ ID NO: 2), PVPGKPGEAV (SEQ ID NO: 3) and NCPVPGKPGEAV (SEQ ID NO: 4) of human TK1; and
a monoclonal antibody, or an antigen-binding fragment thereof, having specificity for a conformation dependent epitope of human TK1; and
the second monoclonal antibody, or the second antigen-binding fragment thereof, is selected from the group consisting of:
a monoclonal antibody, or an antigen-binding fragment thereof, having specificity for GEAVAARKLF (SEQ ID NO: 1) of human TK1;
a monoclonal antibody, or an antigen-binding fragment thereof, having specificity for at least one of NCPVPGKPGE (SEQ ID NO: 2), PVPGKPGEAV (SEQ ID NO: 3) and NCPVPGKPGEAV (SEQ ID NO: 4) of human TK1; and
a monoclonal antibody, or an antigen-binding fragment thereof, having specificity for a conformation dependent epitope of human TK1.

38. The method according to claim 22, wherein the first monoclonal antibody, or the first antigen-binding fragment thereof, is selected from the group consisting of:
a monoclonal antibody, or antigen-binding fragment thereof, having
a variable heavy (VH) domain complementarity determining region 1 (CDR1) having amino acid sequence SEQ ID NO: 5;
a VH domain CDR2 having amino acid sequence SEQ ID NO: 6;
a VH domain CDR3 having amino acid sequence SEQ ID NO: 7;
a variable light (VL) domain CDR1 having amino acid sequence SEQ ID NO: 8;
a VL domain CDR2 having amino acid sequence SEQ ID NO: 9; and
a VL domain CDR3 having amino acid sequence SEQ ID NO: 10;
a monoclonal antibody, or antigen-binding fragment thereof, having
a VH domain CDR1 having amino acid sequence SEQ ID NO: 5;
a VH domain CDR2 having amino acid sequence SEQ ID NO: 11;
a VH domain CDR3 having amino acid sequence SEQ ID NO: 12;
a VL domain CDR1 having amino acid sequence SEQ ID NO: 13;
a VL domain CDR2 having amino acid sequence SEQ ID NO: 9; and
a VL domain CDR3 having amino acid sequence SEQ ID NO: 10; or
a monoclonal antibody, or antigen-binding fragment thereof, having
a VH domain CDR1 having amino acid sequence SEQ ID NO: 14;
a VH domain CDR2 having amino acid sequence SEQ ID NO: 15;
a VH domain CDR3 having amino acid sequence SEQ ID NO: 16;
a VL domain CDR1 having amino acid sequence SEQ ID NO: 17;
a VL domain CDR2 having amino acid sequence SEQ ID NO: 18; and
a VL domain CDR3 having amino acid sequence SEQ ID NO: 19.

39. The method according to claim 22, wherein the second monoclonal antibody, or the second antigen-binding fragment thereof, is a selected from the group consisting of:
a monoclonal antibody, or antigen-binding fragment thereof, having
a variable heavy (VH) domain complementarity determining region 1 (CDR1) having amino acid sequence SEQ ID NO: 5;
a VH domain CDR2 having amino acid sequence SEQ ID NO: 6;
a VH domain CDR3 having amino acid sequence SEQ ID NO: 7;
a variable light (VL) domain CDR1 having amino acid sequence SEQ ID NO: 8;
a VL domain CDR2 having amino acid sequence SEQ ID NO: 9; and
a VL domain CDR3 having amino acid sequence SEQ ID NO: 10;
a monoclonal antibody, or antigen-binding fragment thereof, having
a VH domain CDR1 having amino acid sequence SEQ ID NO: 5;
a VH domain CDR2 having amino acid sequence SEQ ID NO: 11;
a VH domain CDR3 having amino acid sequence SEQ ID NO: 12;
a VL domain CDR1 having amino acid sequence SEQ ID NO: 13;
a VL domain CDR2 having amino acid sequence SEQ ID NO: 9; and
a VL domain CDR3 having amino acid sequence SEQ ID NO: 10; or
a monoclonal antibody, or antigen-binding fragment thereof, having
a VH domain CDR1 having amino acid sequence SEQ ID NO: 14;
a VH domain CDR2 having amino acid sequence SEQ ID NO: 15;
a VH domain CDR3 having amino acid sequence SEQ ID NO: 16;
a VL domain CDR1 having amino acid sequence SEQ ID NO: 17;
a VL domain CDR2 having amino acid sequence SEQ ID NO: 18; and
a VL domain CDR3 having amino acid sequence SEQ ID NO: 19.

40. The method according to claim 22, wherein one of the first monoclonal antibody, or the first antigen-binding fragment thereof, and the second monoclonal antibody, or the second antigen-binding fragment thereof, is immobilized to a solid support or intended to be immobilized to the solid support.

41. The method according to claim 22, wherein the kit is an enzyme-linked immunosorbent assay (ELISA) kit.

42. The method according to claim 22, wherein determining the amount of CA 125 or HE4 comprises determining the amount of CA 125 or HE4 in the serum or plasma sample or the another serum or plasma sample from the female human subject using an electrochemiluminescent immunoassay (ECLIA) kit.

43. The method according to claim 22, further comprising selecting an anti-cancer treatment for the female human subject based on the predicted ovarian cancer risk.

44. The method according to claim 22, further comprising selecting a patient surveillance schedule for the female human subject based on the predicted ovarian cancer risk for the female human subject.

45. A method for determining ovarian cancer biomarkers for a female human subject comprising:
determining an amount of serum thymidine kinase 1 (STK1) material in a serum or plasma sample from the female human subject using a kit comprising a first monoclonal antibody, or a first antigen-binding fragment thereof, specifically binding to a serum form of human TK1 and a second monoclonal antibody, or a second antigen-binding fragment thereof, specifically binding to the serum form of human TK1; and
determining an amount of carcinoma antigen 125 (CA 125) or human epididymis protein 4 (HE4), but not both of CA 125 and HE4, in the serum or plasma sample or another serum or plasma sample from the female human subject.
